(19) United States
(12) Patent Application Publication
(10) Pub. No.: US 2020/0188001 A1
(43) Pub. Date: Jun. 18, 2020

(54) BONE FIXATION AND REPAIR SYSTEM

(71) Applicant: Zimmer Biomet CMF and Thoracic, LLC, Jacksonville, FL (US)

(72) Inventors: Andrew Charles Davison, Downingtown, PA (US); Kenneth Carlton Kremer, Hershey, PA (US)

(21) Appl. No.: 16/714,057

(22) Filed: Dec. 13, 2019

Related U.S. Application Data
(60) Provisional application No. 62/779,747, filed on Dec. 14, 2018.

(51) Int. Cl. A61B 17/80; A61B 17/86

(52) U.S. Cl. CPC A61B 17/8076; A61B 2017/00004; A61B 2017/8655; A61B 17/86

(57) ABSTRACT

A bone fixation and repair system for a fracture in a bone includes a bone plate having at least one opening. The system includes at least one elongated flexible element and a fastener assembly configured to fit within the at least one opening. The fastener assembly has a) a fastener and b) a locking cap configured to engage the shaft of the fastener.

### CLAIM OF PRIORITY

This application claims the benefit of U.S. Provisional Patent Application Ser. No. 62/779,747, filed on Dec. 14, 2018, the benefit of priority of which is claimed hereby, and which is incorporated by reference herein in its entirety.

### TECHNICAL FIELD

The present disclosure is directed to a bone fixation and a repair system, and in particular to a bone fixation and repair for rib fractures.

### BACKGROUND

Ribs fractures are difficult to heal. Conventional repair procedures are invasive and typically require large incisions in the thoracic cavity to access the fracture site and implant the desired bone plates. Such procedures delay healing. Developments have been made to make rib fixation less invasive, such as those set forth in U.S. Pat. No. 8,728,133 and U.S. Patent App. Pub. No. 2014/0277175. Work continues to improve on minimally invasive bone fixation and repair systems for rib fractures.

### SUMMARY

There is a need to improve clinical outcomes for rib fixation and repair. An embodiment of the present disclosure is a bone fixation and repair system for a fracture in a bone, such as a rib. The system includes a bone plate having at least one opening, such as an elongated slot or other type of opening. The system includes at least one elongated flexible element and a fastener assembly configured to fit within the at least one opening. The elongated flexible element may, in some embodiments, by integrated with the fastener to define a tethered fastener assembly in certain embodiments, the fastener assembly has a fastener and a locking cap configured to engage the fastener.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings illustrative embodiments of the disclosure. It should be understood, however, that the application is not limited to the precise arrangements and instrumentalities shown. In the drawings:

FIG. 1 is a perspective view of a bone fixation and repair system according to an embodiment of the present disclosure;

FIG. 2 is a cross-section of the bone fixation and repair system taken along line 2-2 in FIG. 1;

FIG. 3A is a top perspective view of a bone plate in the system shown in FIGS. 1 and 2;

FIG. 3B is a top view of the bone plate shown in FIG. 3A;

FIG. 3C is a cross-section of the bone plate taken along line 3-3 in FIG. 3B;

FIG. 4A is a perspective view of a bone plate according to another embodiment of the present disclosure;

FIG. 4B is a side view of the bone plate shown in FIG. 4A;

FIG. 5A is a top view of a bone plate according to another embodiment of the present disclosure;

FIG. 5B is a top view of a bone plate according to another embodiment of present disclosure;

FIG. 5C is a top view of a bone plate according to another embodiment of the present disclosure;

FIG. 5D is a top view of a bone plate according to another embodiment of the present disclosure;

FIG. 5E is a top view of a bone plate according to another embodiment of the present disclosure;

FIG. 6 is a perspective view of a bone fixation and repair system according to another embodiment of the present disclosure;

FIG. 7 is a cross-section of the bone fixation and repair system taken along line 7-7 in FIG. 6;

FIG. 8 is a cross-section of the bone fixation and repair system taken along line 8-8 in FIG. 6;

FIG. 9 is a perspective view of a tethered fastener assembly of the bone fixation system illustrated in FIGS. 1-8;

FIG. 10A is a side view of a fastener in the tethered fastener assembly shown in FIG. 9;

FIG. 10B is a cross-sectional view of the fastener shown in FIG. 9;

FIG. 11A is a perspective view of an instrument assembly and a tethered fastener assembly according to an embodiment of the present disclosure;

FIG. 11B is a perspective view of the fastener in the tethered fastener assembly shown in FIG. 11A;

FIG. 11C is an exploded perspective view of the instrument assembly and tethered fastener assembly shown in FIG. 11A;

FIG. 11D is an end view of an outer driving instrument of the driving assembly shown in FIGS. 11A and 11C;

FIG. 11E is an end view of an inner driving instrument of the driving assembly shown in FIGS. 11A and 11C;

FIG. 12A is a side view of a fastener of the tethered fastener assembly shown in FIGS. 11A-11C;

FIG. 12B is an end view of the fastener shown in FIG. 12A;

FIG. 13A is a perspective view of a bone fixation and repair system according to another embodiment of the present disclosure;

FIG. 13B is a cross-section of the bone fixation and repair system taken along line 13-13 in FIG. 13A;

FIG. 14A is a perspective view of a fastener in the system shown in FIGS. 13A and 13B;

FIG. 14B is a side view of a fastener in the system shown in FIG. 14A;

FIG. 14C is a cross-sectional view of the fastener shown in FIGS. 14A and 14B;

FIG. 15A is a perspective view of a fastener according another embodiment of the present disclosure;

FIG. 15B is a side view of a fastener shown in FIG. 15A;

FIG. 16A is a perspective view of a fastener according another embodiment of the present disclosure;

FIG. 16B is a side view of a fastener shown in FIG. 16A;

FIG. 17A is a perspective view of a fastener according another embodiment of the present disclosure;

FIG. 17B is a side view of a fastener shown in FIG. 17A;

FIG. 18A is a perspective view of a fastener according another embodiment of the present disclosure;

FIG. 18B is a side view of a fastener shown in FIG. 18A;

FIG. 19A is a perspective view of a bone fixation and repair system according to another embodiment of the present disclosure;

FIG. 19B is a bottom view of the bone fixation and repair system shown in FIG. 19A;

FIG. 19C is a detailed view of the bone fixation and repair system shown in FIGS. 19B;

FIG. 20A is a perspective view of a fastener for the system shown in FIGS. 19A-19C, according another embodiment of the present disclosure;

FIG. 20B is a side view of the fastener for the system shown FIG. 20A;

FIG. 21A is a perspective view of a fastener for the system shown in FIGS. 19A-19C, according another embodiment of the present disclosure;

FIG. 21B is a side view of the fastener for the system shown FIG. 21A;

FIG. 22 is a perspective sectional view of a bone fixation and repair system according to another embodiment of the present disclosure;

FIG. 23 is a perspective sectional view of the bone fixation and repair system shown in FIG. 22, illustrating suture tails of a suture assembly cut;

FIG. 24 is a perspective sectional view of the bone fixation and repair system shown in FIG. 22, illustrating suture tails tied to secure a locking cap in place;

FIG. 25A is a perspective view of a fastener for the bone fixation and repair system shown in FIG. 22;

FIG. 25B is a perspective view of a fastener for the bone fixation and repair system shown in FIG. 22;

FIG. 26A is a perspective view of a locking cap for the bone fixation and repair system shown in FIG. 22;

FIG. 26B is the side view of the locking cap shown in FIG. 26A;

FIG. 27 is a side view of a bone fixation and repair system according to another embodiment of the present disclosure;

FIG. 28 is a perspective view of a movable toggle used in the bone fixation and repair system shown in FIG. 27;

FIG. 29A is a side view of a drive assembly of a bone fixation and repair system according to another embodiment of the present disclosure;

FIG. 29B is another side view of the drive assembly of a bone fixation and repair system shown in FIG. 29A;

FIG. 30A is a perspective view of a bone fixation and repair system according to another embodiment of the present disclosure;

FIG. 30B is a cross-section of the bone fixation and repair system taken along line 30-30 in FIG. 30A;

FIG. 30C is a detailed view of cross-section of the bone fixation and repair system shown in FIG. 30B;

FIG. 30D is a perspective view of a fastener and elongated flexible element for the bone fixation and repair system shown in FIGS. 30A-30C;

FIG. 31A is a perspective view of a bone fixation and repair system according to another embodiment of the present disclosure;

FIG. 31B is a cross-section of the bone fixation and repair system taken along line 31-31 in FIG. 31A;

FIG. 31C is a detailed view of cross-section of the bone fixation and repair system shown in FIG. 31B;

FIG. 31D is a perspective view of a fastener and elongated flexible element for bone fixation and repair system shown in FIGS. 31A-31C;

FIG. 32A is a side view of a fastener for the bone fixation and repair system shown in FIGS. 30A-31C;

FIG. 32B is a sectional view of a fastener shown in FIG. 32A.

FIG. 33 is a perspective view of instrumentation used in the bone fixation system shown in FIGS. 1-32B, according to an embodiment of the present disclosure;

FIG. 34 is a side view of the instrumentation shown in FIG. 33;

FIG. 35 is a perspective exploded view of the instrumentation shown in FIG. 33;

FIG. 36A is a perspective view of a clamping device shown in FIG. 33;

FIG. 36B is a side view of the clamping device shown in FIG. 36A;

FIGS. 36C and 36D are front and rear views, respectively, of the clamping device shown in FIG. 36A; and

FIG. 37 is another perspective view of the clamping device shown in FIG. 36A.

### DETAILED DESCRIPTION OF ILLUSTRATIVE EMBODIMENTS

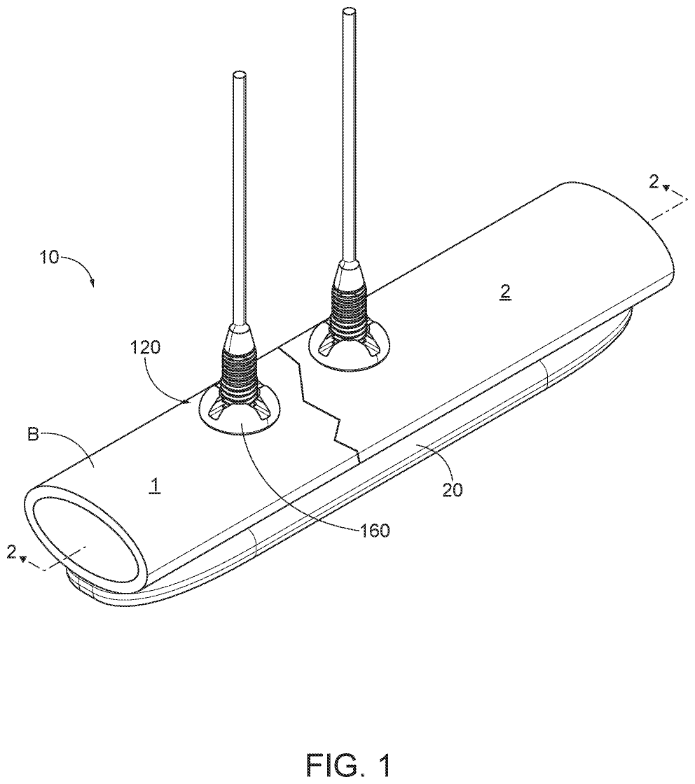

Embodiments of the present disclosure include systems and methods for bone fixation and repair, and in particular, for a bone fixation and repair of rib fractures. FIG. 1-4B illustrates an embodiment of a system 10 for repairing a fracture in a bone B having a first bone segment 1 and a second bone segment 2. The system 10 includes a bone plate 20 that is elongated along a central longitudinal axis, one or more tethered fastener assemblies 120, and one or more corresponding locking caps 160. As shown, the bone plate 20 and has a first end, a second end spaced from the first end along the central longitudinal axis, a first and second side member on opposite sides of the central longitudinal axis, and an elongated slot that extends through the bone plate 20 along a depth D that is perpendicular to the central longitudinal axis. The bone plate 20 defines a) a length L that extends from the first end to the second. The length L is perpendicular to the depth D, and b) a width W that extends from the first side member to the second side member where the width W is perpendicular to the length L and the depth D. As illustrated, the elongated slot extends along a substantial entirety of the length L of the bone plate 20.

As illustrated in FIGS. 1-3C, the bone plate 20 includes a first side member and a second side member. The first and second side members each define a bottom surface, a top surface spaced from the bottom surface along the depth, and an internal engagement surface that extends from the bottom surface toward the central longitudinal axis. The top surface of the first side member and the second side member may be curved in order to receive a curved surface of a bone B. In one example, the internal engagement surfaces a) are non-orthogonal to the depth and b) are opposed to each other to partially define the elongated slot. The internal engagement surfaces may extend along an entirety of the first side member and the second side member. In one example, the internal engagement surfaces are curved with respect to the depth D. In one another example, the internal engagement surfaces are flat and angled with respect to the depth D. In another embodiment not shown, the first and second side members may include first and second ridges along the top of the first and second side members, respectively. The ridges are configured to engage the bone B and help facilitate engagement with the bone.

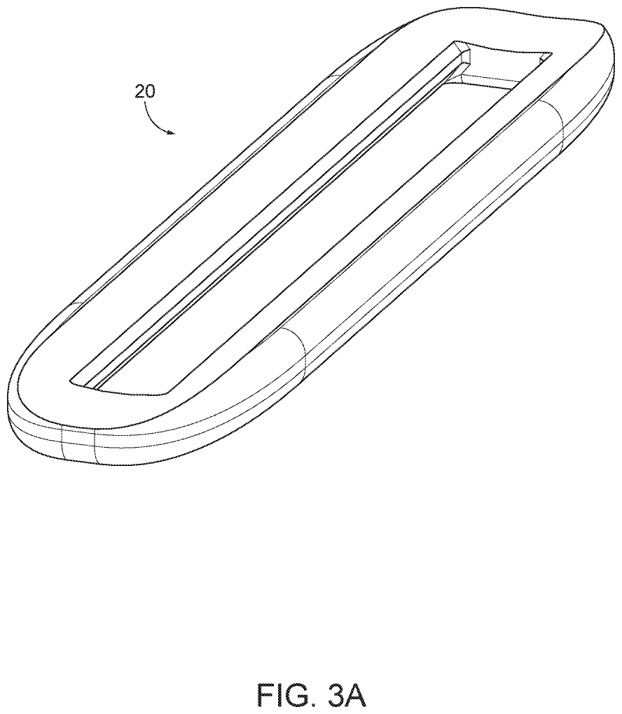

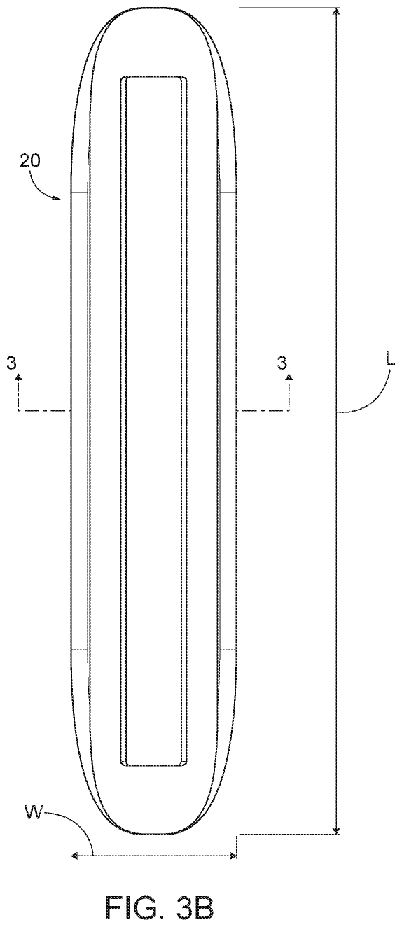

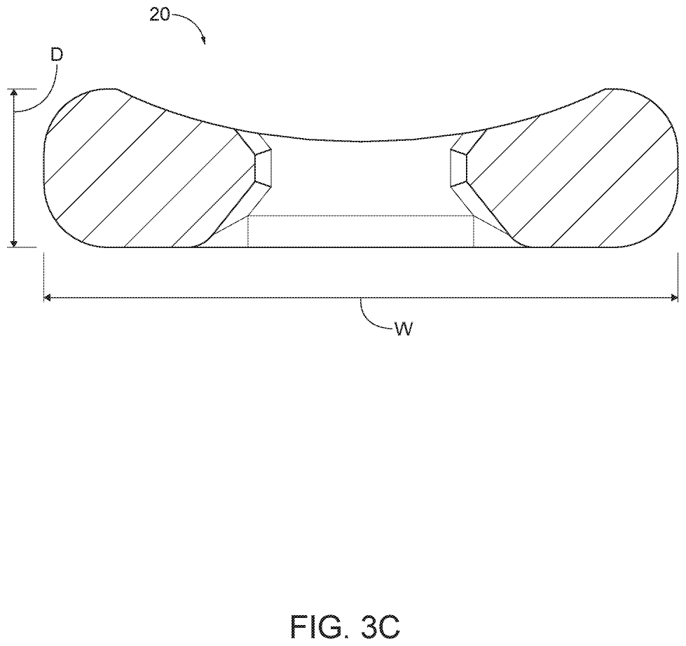

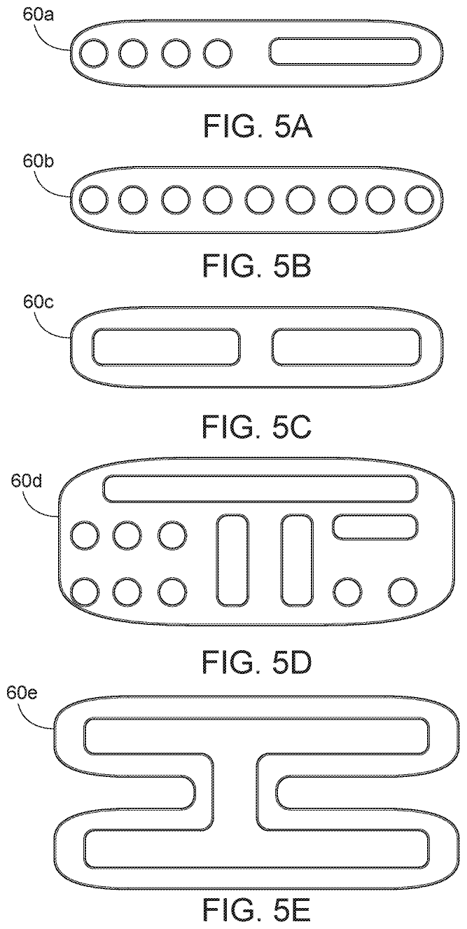

The bone fixation systems described herein may be use a wide range of bone plates configurations. For instance, the bone plate 20 may have a single elongated slot as shown in FIGS. 3A-3C. In another example as shown in FIG. 5A, the bone plate 60a may further comprises at least two openings that extend through the bone plate along the depth, and at least one elongated slot. In another example as shown in FIG. 5B, the bone plate 60b may further comprise sets of circular openings that extend through the bone plate. However, as shown in FIG. 5C, a bone plate 60c may comprise one or more elongated slots. In another example as shown in FIG. 5D, a bone plate 60d has a plurality of elongated slots that have a different size and orientation with respect to the each other. The bone plate 60d may further include a plurality of openings in addition to the plurality of elongated slots.

In another example as shown in FIG. 5E, the bone plate 60e includes a first segment including the first side member, the second side member, and the first elongated slot. The bone plate 60e may also include a second segment having a first side member, a second side member, a second elongated slot, where the first side member and the second side member of the second segment is substantially similar to the first side member and the second side member of the first segment. The bone plate 60e may also include a bridge segment that couples the first segment to the second segment. The bridge segment defines a bridge slot that is open to the first and second elongated slots. In this example, the tethered fastener assemblies 120 are movable along any portion of the first elongated slot, the second elongated slot, and the bridge slot.

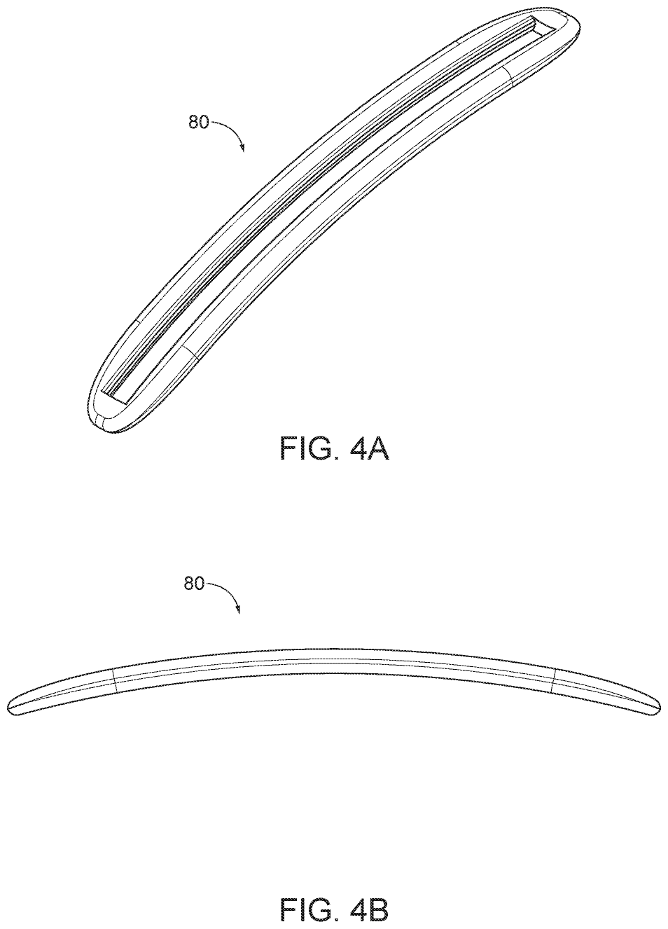

In another embodiment, as shown in FIGS. 4A, 4B, a bone plate 80 may be curved along the central longitudinal axis. The bone plate 80 includes similar features to the bone plate 20 described above and illustrated in FIGS. 1-3C expect the bone plate 80 is curved.

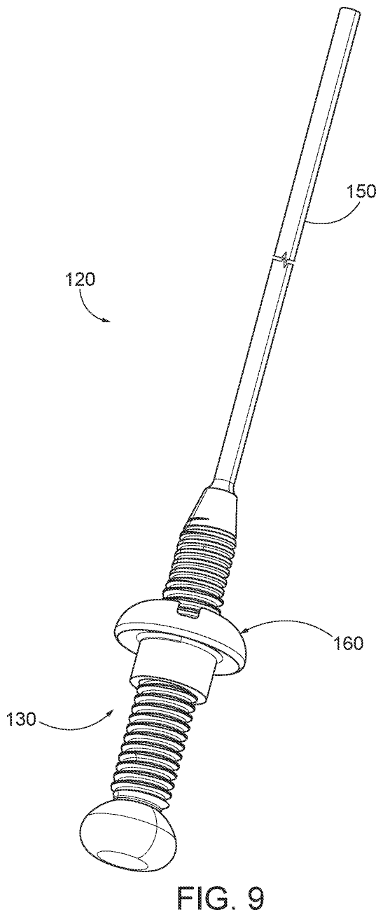

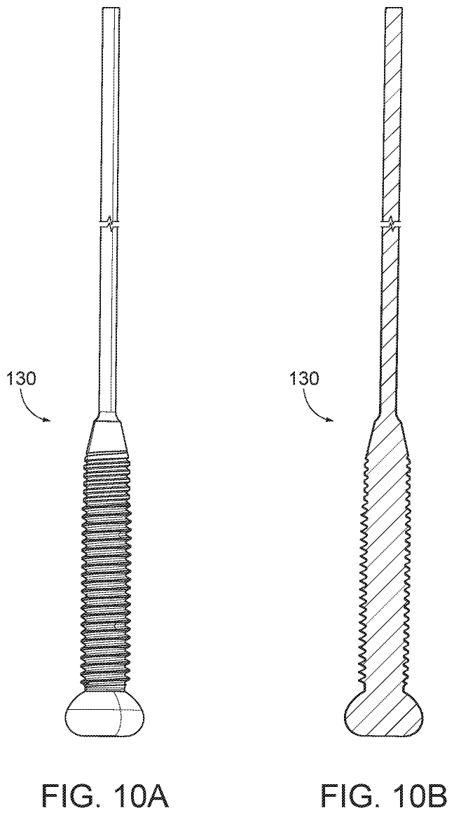

Turning to FIGS. 9-10B, the bone fixation system 10 includes a plurality of a tethered fastener assemblies 120 configured to concurrently fit within and slide along any portion of the elongated slot in the bone plates described herein. As illustrated, the tethered fastener assembly has a fastener 130, an elongated flexible element 150 integrated with the fastener, and a locking cap 160. In accordance with the illustrated embodiment, a head of the fastener 130 defines a distal end of the assembly 120 and the elongated flexible element 150 defines a proximal end of the assembly 120 such that the tethered fastener assembly 120 is an integrated unit.

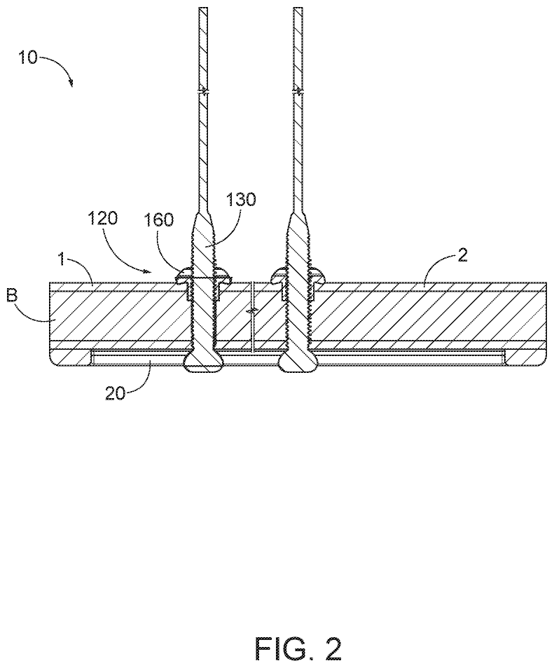

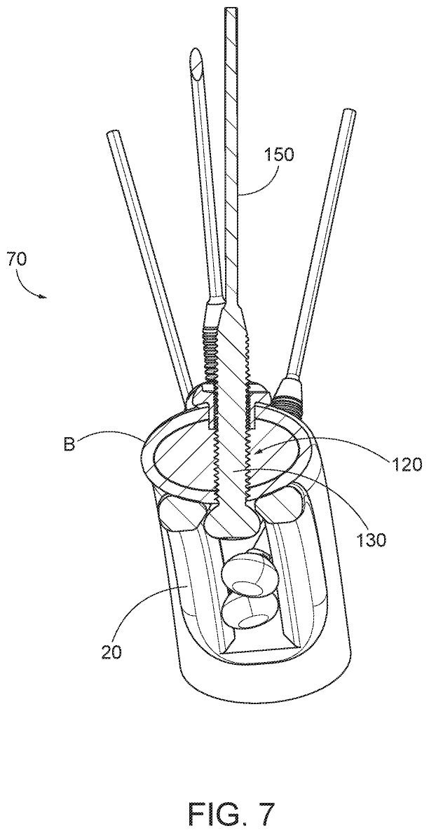

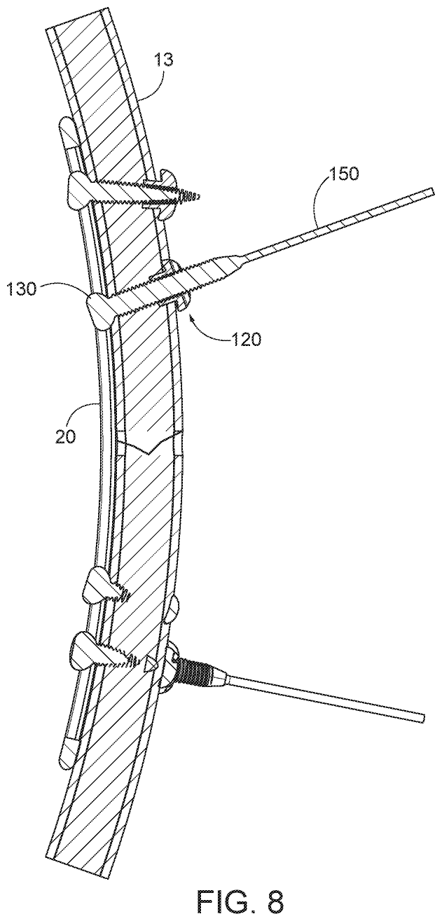

The fastener 130 has a head, a shaft that extends from the head along a central fastener axis, and an elongated flexible element 150 that is attached to and extends from the shaft. The head defines a smooth outer surface configured to movably engage the internal engagement surfaces of the bone plate 20, such that the fastener 130 is rotatable about multiple axes when the smooth outer surface of the head is in contact the internal engagement surfaces of the bone plate 20. See FIGS. 2, 7 and 8. In one example, the smooth outer surface of the head is curved with respect to the central fastener axis. In another example, the smooth outer surface of the head is linear and angled with respect to the central fastener axis. The elongated slot of the bone plate 20 is sized to permit the shaft to pass therethrough but inhibit the head to pass therethrough. As illustrated in FIGS. 9-10B, at least a portion of the shaft is threaded.

Referring to FIGS. 9-10B, the elongated flexible element 150 may be any length of material. For instance, the elongated flexible element 150 may include, but is not limited to a cable, a suture, suture assembly, wire, or any other length of material. The elongated flexible element 150 may be attached to the fastener 130 or it could be monolithic with the fastener 130. The elongated flexible element 150 may have a diameter between 0.5 mm and 10 mm. The elongated flexible element may have a length, (measured form the distal tip of the shaft to an end of the elongated flexible element, between 250 mm and 2250 mm. In one example, the elongated flexible element 150 is between 250 mm and 750 mm. In one example, the elongated flexible element is between 500 mm and 1000 mm. In one example, the elongated flexible element is between 750 mm and 1250 mm. In one example, the elongated flexible element has a length between 1000 mm and 1500 mm. In one example, the elongated flexible element has a length between 1250 mm and 1750 mm. In another example, the elongated flexible element has a length that is between 1500 and 2000 mm. In another example, the elongated flexible element has a length that is between 1750 and 2250 mm. In some instances, the elongate flexible element may be stainless steel, nitinol, or other biomedical material suitable for implantation in a human body. Alternatively, the elongated flexible element 150 may be polymeric. In some instances, the elongated flexible element is bioabsorbable.

The locking cap 160 is configured to fix the fastener 130 in the elongated slot of the bone plate 20 when a) the bone plate 20 is in contact with the bone, and b) the locking cap 160 is engaged with the shaft and in contact with the bone. Furthermore, the locking cap 160 is configured to slide along the elongated flexible element 150 into engagement with the shaft. The locking cap 160 includes an internal channel and internal threads that are configured for threaded engagement with the threaded shaft of the fastener 130.

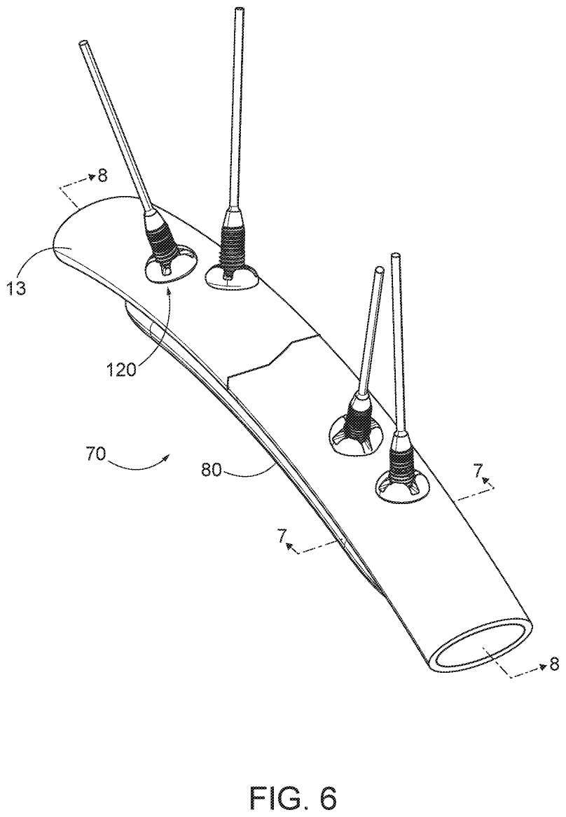

Referring back to FIGS. 6-8, an embodiment of the present disclosure includes a bone fixation system 70 that includes a curved bone plate 80 and multiple sets of tethered fastener assemblies 120 that lock the bone plate 80 in place the bone B, as described above.

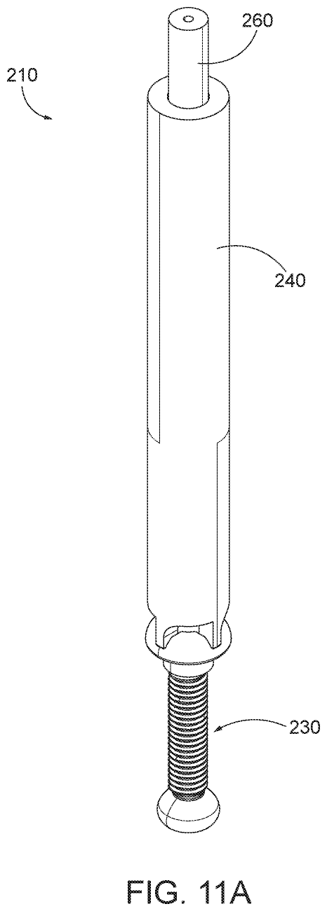

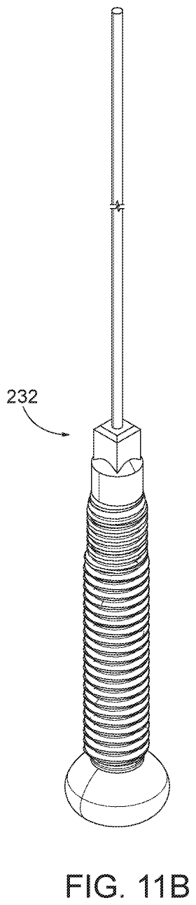

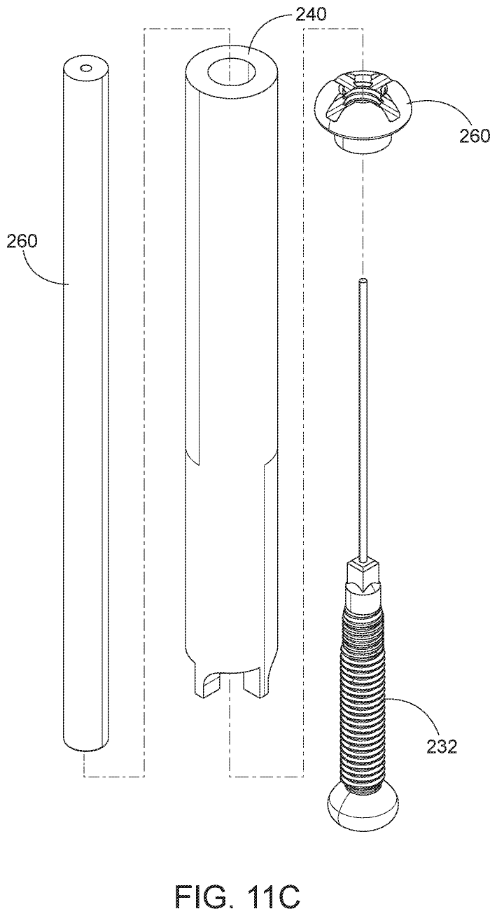

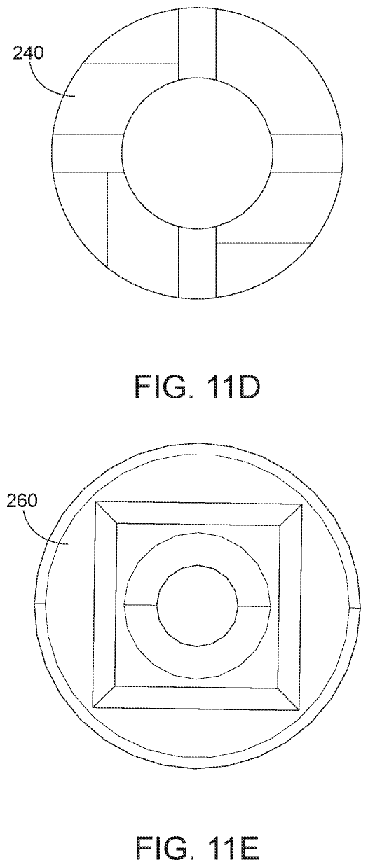

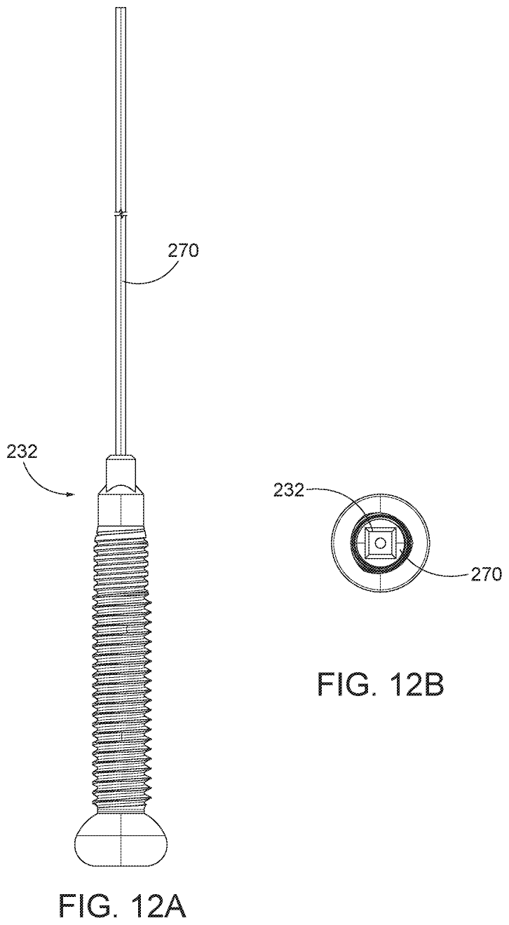

As shown in FIGS. 11-12B, another embodiment of the disclosure is a tethered fastener assembly 230 is configured to engage with a driving instrument assembly 210. The tethered fastener assembly 230 may be used with any of the bone plates described in the present disclosure. The fastener assembly 230 includes a fastener 232 and an elongated flexible element 270. As illustrated, the shaft of the fastener 230 extends from the head to a shaft tip, wherein the shaft tip defines a tip engagement feature configured to receive a torque. In one example of this embodiment, the shaft is threaded. However, the shaft may have other engagement features, such as ratchet teeth.

Referring to FIGS. 11-12B, the driving instrument assembly 210 may include an outer driving instrument 240 and an inner driving instrument 260. The outer driving instrument 240 defines a proximal end and a distal end opposite the proximal end, and an internal channel that extends from the proximal end to the distal end. The distal end defines an engagement member configured to engage an upper surface of the locking cap so as to drive the locking cap into engagement with the shaft of the fastener.

Continuing with FIGS. 11-12B, the internal driving instrument 260 has a back end, a driving end, and an outer surface. The outer surface of the internal driving instrument 260 is slidable within the internal channel of the outer driving instrument 240. The driving end further defines an instrument engagement feature that mates with the tip engagement feature such that the inner driving instrument 260 is configured to apply torque to the shaft tip of the fastener when the instrument engagement is mated with the tip engagement feature.

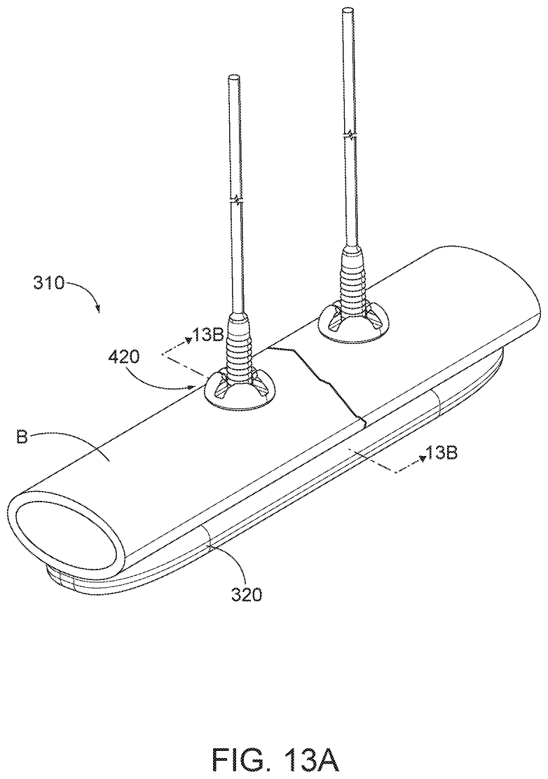

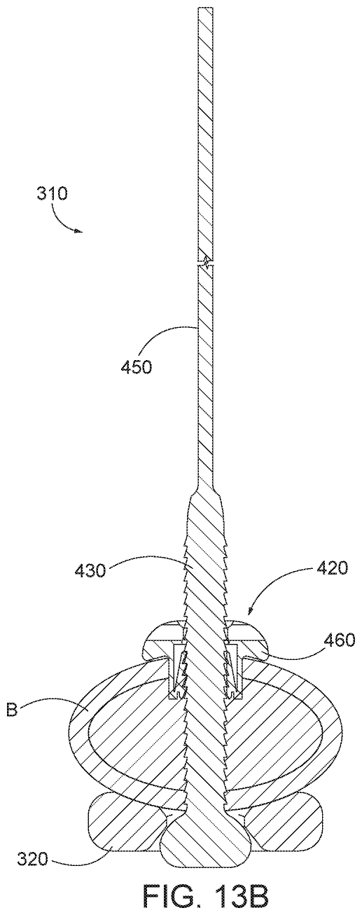

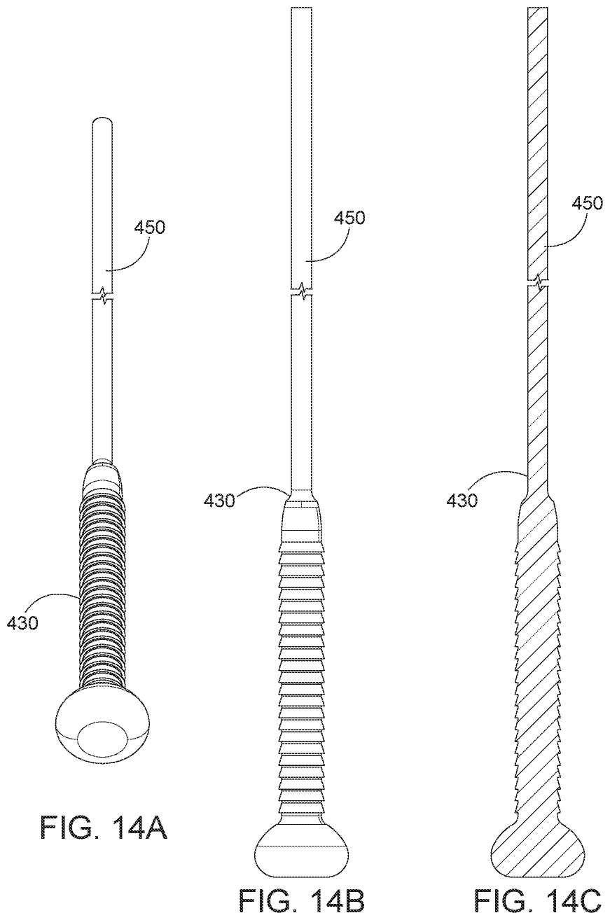

Another embodiment of a bone fixation and repair system 310 is shown in FIGS. 13A-14C. The system 310 shown includes a bone plate 320 configured to be placed against a bone B. In the embodiment shown in FIGS. 13A-14C, the bone plate 320 includes at least one opening that extends through the bone plate 320. The opening may be a circular opening or one or more elongated slots as described above in the bone plates 20, 60a-60e, and 80 and illustrated in FIGS. 1-5D.

The system 310 shown in FIGS. 13A-14C includes a plurality of a tethered fastener assemblies 420 configured to secure the bone plate 320 to the bone. The tethered fastener assembly 420 has a fastener 430, an elongated flexible element 450, and a locking cap 460. A head of the fastener 430 defines a distal end of the assembly and the elongated flexible element 450 defines a proximal end of the assembly such that the tethered fastener assembly 430 is an integrated unit, as described above.

In the embodiment shown FIGS. 13A-14C, the fastener 430 has a head, a shaft that extends from the head along a central fastener axis, and an elongated flexible element 450 attached to and extending from the shaft. In this embodiment, the shaft of the fastener defines external ratchet teeth.

Referring to FIGS. 13A-14C, the locking cap 460 is configured to be attached to the fastener 430. As shown, the locking cap 460 has a top, a bottom, an inner surface, and an inner opening defined by the inner surface. The inner surface defines internal ratchet teeth that are configured to engage the external ratchet teeth of the shaft so as to lock the locking cap to the fastener 430.

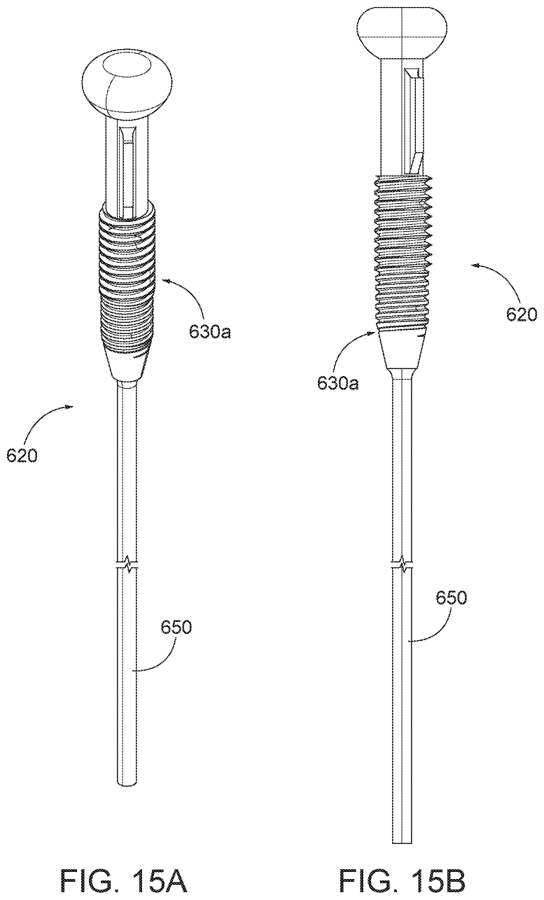

As shown FIGS. 15A-15B, an embodiment of the present disclosure includes a bone fixation and repair system having a bone plate as described herein and configured to be placed against a bone. This particular system is not shown or numbered. Any of the bone plates described in this disclosure may be used with fastener assembly 620 illustrated in FIGS. 15A-15B. For instance, the bone plate may include at least one opening that extends through the bone plate. The opening may be one or more slots. Alternatively, the opening may be one or more circular openings. The system may include at least one tethered fastener assemblies 620 configured to extend through the at least one opening of the bone plate. The tethered fastener assembly 620 includes a fastener 630a having a head, a shaft that extends from the head along a central fastener axis, and an elongated flexible element 650 attached to and extending from the shaft. The shaft of the fastener 630a has 1) a distal portion adjacent the head and has at least one engagement feature, and 2) a proximal portion adjacent to the distal portion such that the distal portion extends from the head to the proximal portion. The locking cap 660 (not shown) is configured to engage the proximal portion of the shaft but not the distal portion of the shaft of the fastener 630a. The locking cap 660 may be similar to other locking caps described in the present disclosure.

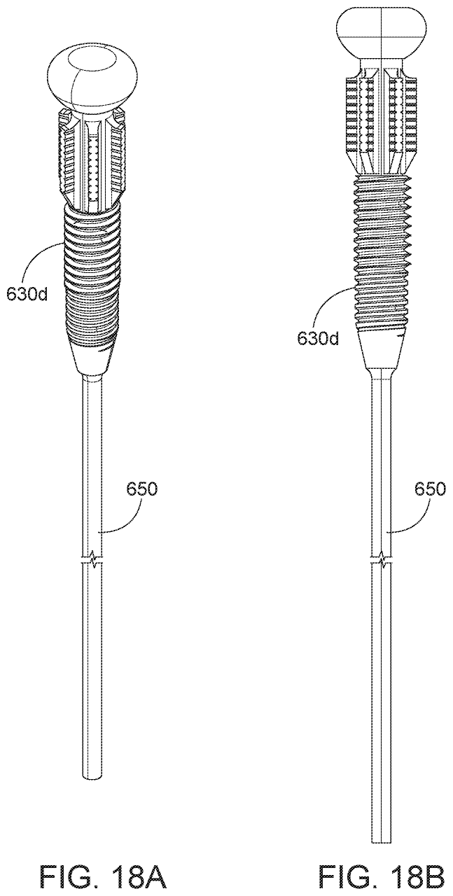

In accordance with the illustrated embodiment shown in FIGS. 15A-18B, the proximal portion of shaft defines external threads and the locking cap is configured to threadably engage the external threads on the proximal portion of the shaft. The distal portion may include a number of different engagement feature configurations. As shown in FIG. 15A-15B, a fastener 630a includes an engagement feature configured as a single keel is illustrated. The keel extends outwardly from the shaft along a radial direction that this perpendicular to the central fastener axis of the shaft. The keel is configured to engage bone and help secure the fastener in place. In the example shown in FIG. 18A-18B, the fastener 603d includes a plurality of keels and each keel extends outwardly from the shaft along a radial direction that this perpendicular to the central axis.

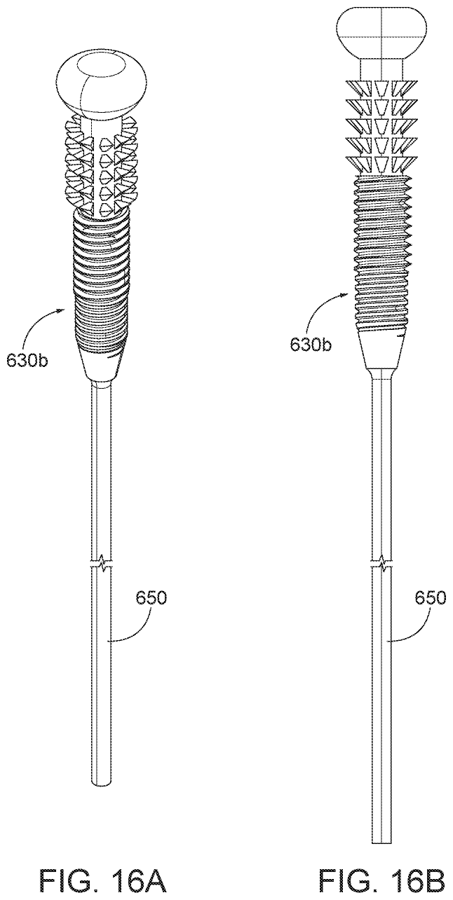

As shown in FIG. 16A-16B, a fastener 630b includes an engagement feature configured as a plurality of separate discrete projections. The discrete projections extend outwardly from the shaft along a radial direction that this perpendicular to the central fastener axis of the shaft. The projections are configured to engage bone. As shown, the projections slope from a surface of the shaft toward the head. A lower surface of the projection is substantially parallel to an end surface of the head. In other words, the lower surface is perpendicular to a central fastener axis.

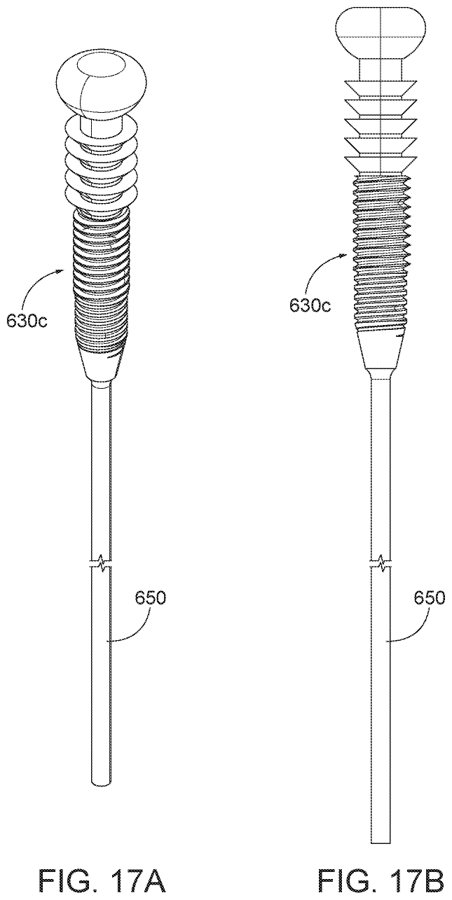

As shown in FIG. 17A-17B, a fastener 630c includes an engagement feature configured as a plurality of ratchet teeth. The ratchet teeth extend outwardly from the shaft along a radial direction that this perpendicular to the central fastener axis of the shaft. The ratchet teeth also circumscribe the shaft to surround the shaft. The ratchet teeth are configured to engage bone.

In the embodiment illustrated in FIGS. 15A-18B, the proximal portion of the fastener may include external threads. In such an embodiment, the locking cap is configured to be attached to the fastener, the locking cap includes top, a bottom, an inner surface, and an inner opening defined by the inner surface. The inner surface defines internal threads that are configured to engage the external threads of the shaft to lock the locking cap to the fastener. In an alternative embodiment, however, the proximal portion of the fastener may include external ratchet teeth and the locking cap includes internal ratchet teeth that are configured to interlock with the external ratchet teeth.

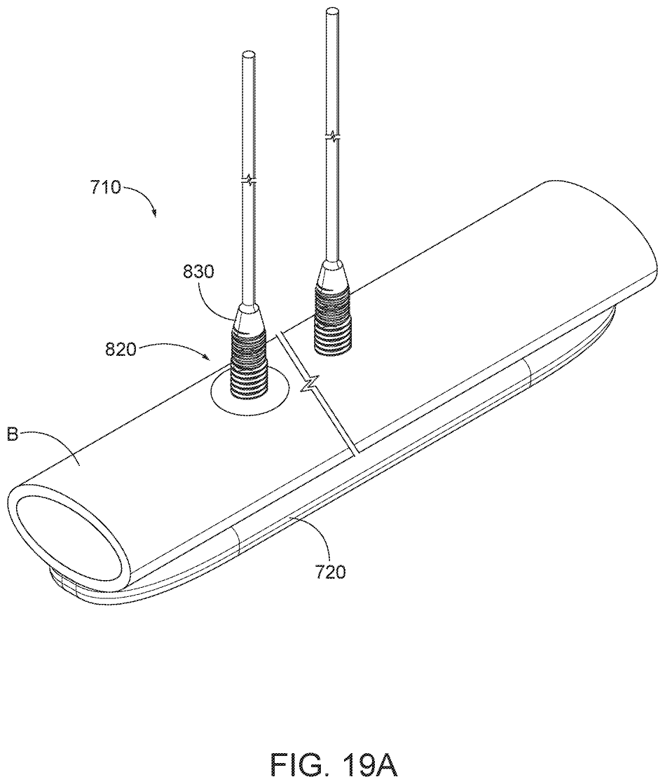

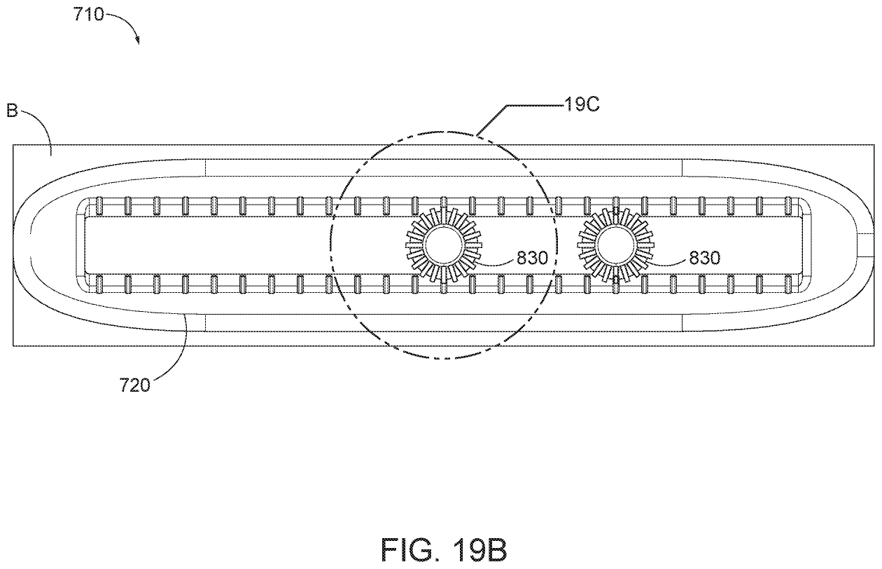

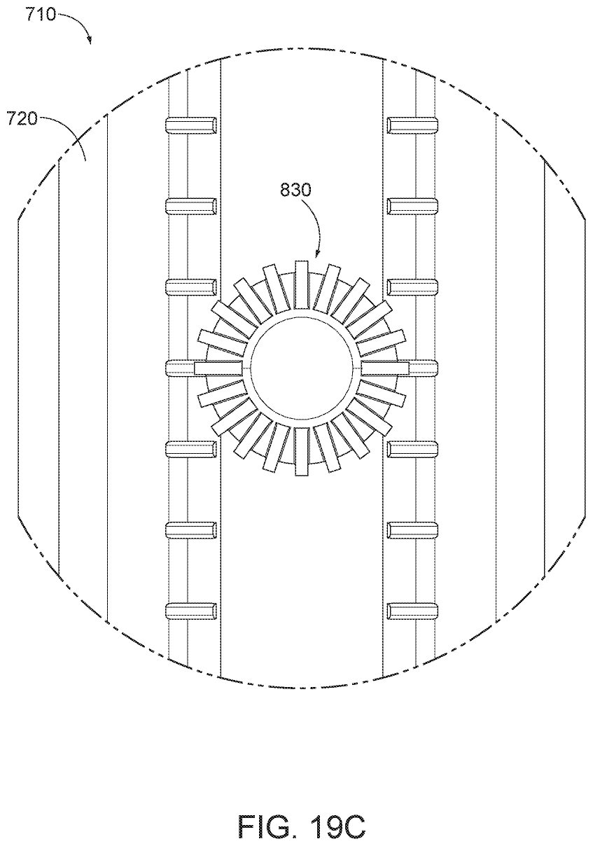

Another embodiment of the present disclosure includes a bone fixation system 710 may with anti-rotation features on the fastener head and/or bone plate as further described herein. As shown in FIGS. 19A-19C, the system 710 include a bone plate 720 configured to be placed against a bone B. The bone plate 720 includes a top configured to face the bone, a bottom opposite the top, at least one opening that extends from the bottom to the top, and a first anti-rotation element disposed along the bottom adjacent the at least one opening. As shown in FIGS. 19C and 19D, the first anti-rotation element may be a plurality of spaced apart grooves disposed along the bottom of the bone plate 720. Alternatively, the first anti-rotation element may be a plurality of spaced apart projections.

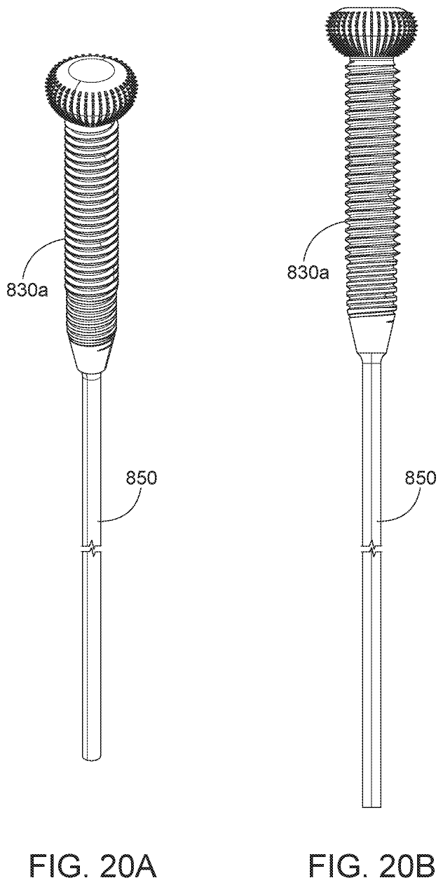

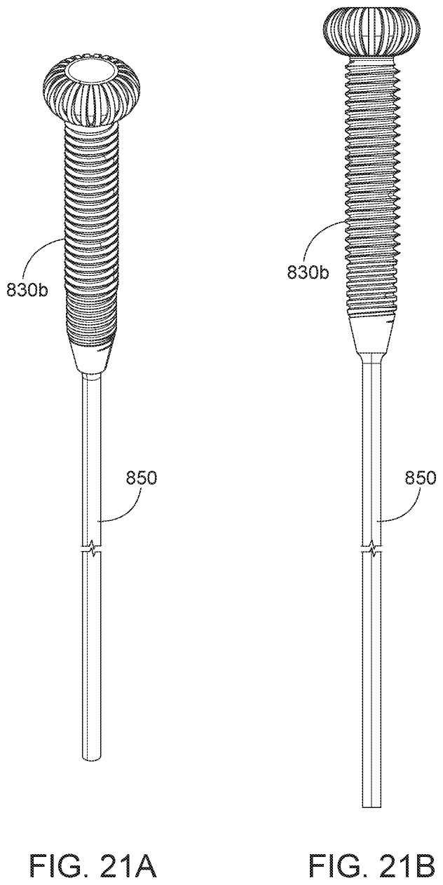

The system 710 also includes at least one tethered fastener assembly 820 configured to extend through the at least one opening of the bone plate 720. The tethered fastener assembly 820 has a fastener 830 having a head, a shaft that extends from the head along a central fastener axis, and an elongated flexible element 850 attached to and extending from the shaft. A locking cap (not shown) as described herein may engage the fastener 830. As illustrated, the head defines a distal end of the assembly and the elongated flexible element 850 defines a proximal end of the assembly such that the tethered fastener assembly is an integrated unit. The head has a second anti-rotation element configured to engage the first anti-rotation element of the bone plate 720. When the fastener is fully seated in opening of the bone plate 20, the first and second anti-rotation elements interlock so that the fastener 830 is inhibited from rotation with respect to the bone plate 720. As shown in FIGS. 20A and 20B, the second anti-rotation element of fastener 830a is plurality of linear ridges that extend outwardly from the head from a proximal surface of the head toward the shaft and each ridge is sized to engage each move of the plate 720. As shown in FIGS. 21A and 21B, the second anti-rotation element of fastener 830b is plurality of linear ridges that extend outwardly from the head from a proximal surface of the head toward the shaft and each ridge is sized to engage each groove of the plate 720.

Furthermore, the fastener in FIGS. 20A-21B may be threaded and the locking cap is configured to threadably engage the shaft. Alternatively, the shaft defines external ratchet teeth and the locking cap defines internal ratchet teeth configured to interlock with the external ratchet teeth. In addition, or alternatively, the fastener may include bone engagement features on the distal portion of the shaft. Such engagement features may include one of: a) a keel configured to engage bone; b) a plurality of keels, c) a plurality of discrete projections that extend outwardly away from the shaft, or d) ratchet teeth.

In accordance with the illustrated embodiment, the elongated flexible element 150 may be any length of material. For instance, the elongated flexible element 150 may include, but is not limited to a cable, a suture, suture assembly, wire, or any other length of material. In one example, the elongated flexible element is a suture assembly. The suture assembly may include a pair suture tails that configured to secure the locking cap in place with respect to the head of the fastener assembly.

Another embodiment of the present disclosure (not shown) includes a bone fixation and repair system includes a bone plate having a top configured to face the bone, a bottom opposite the top, at least one opening that extends from the bottom to the top, and an anti-rotation element disposed along the bottom adjacent the at least one opening. The system also includes at least one tethered fastener assembly configured to extend through the at least one opening. The tethered fastener assembly has a fastener having a head, a shaft that extends from the head along a central fastener axis, and an elongated flexible element attached to and extending from the shaft. When the fastener is fully seated in the at least one opening, the head and the anti-rotation element engage so that the fastener is inhibited from rotation with respect to the bone plate. In accordance with the illustrated embodiment, the anti-rotation element in the bone plate may be is a plurality of spaced apart grooves disposed along the bottom of the bone plate. In another example, the anti-rotation element may include is a plurality of spaced apart ridges disposed along the bottom of the bone plate. Alternatively, the anti-rotation element is a plurality of spaced apart projections disposed along the bottom of the bone plate. The locking cap is configured to engage the shaft.

In another embodiment of the present disclosure (not shown), a bone fixation and repair system includes a bone plate having a top configured to face the bone, a bottom opposite the top, and at least one opening that extends from the bottom to the top. The bone plate in this embodiment may not include a specific anti-rotation element as described above. However, the system also includes at least one tethered fastener assembly configured to extend through the at least one opening. The tethered fastener assembly has a fastener having a head, a shaft that extends from the head along a central fastener axis, and an elongated flexible element attached to and extending from the shaft. Furthermore, the head includes an anti-rotation element configured to engage the bottom of the bone plate such that when the fastener is fully seated in the at least one opening, the anti-rotation element and the bottom of the bone plate interlock so that the fastener is inhibited from rotation with respect to the bone plate. As with other embodiments, a locking cap may be used to engage the shaft in any embodiment described above, the anti-rotation elements may be ridges, projections, grooves or any surface the provide an interference fit.

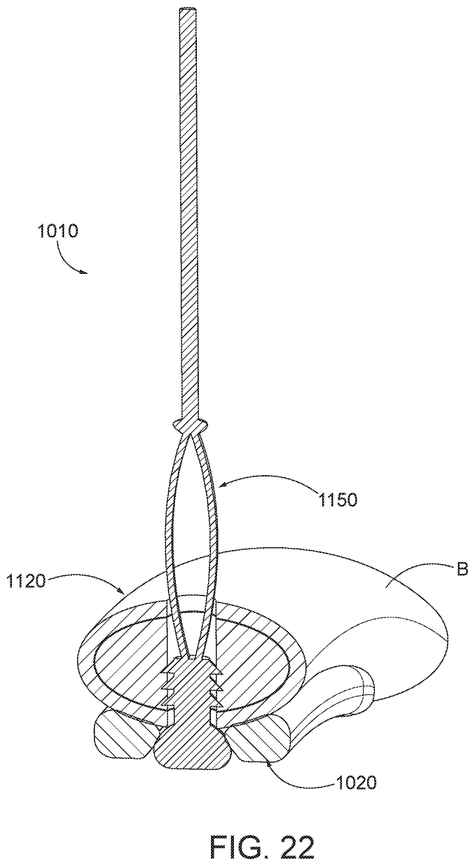

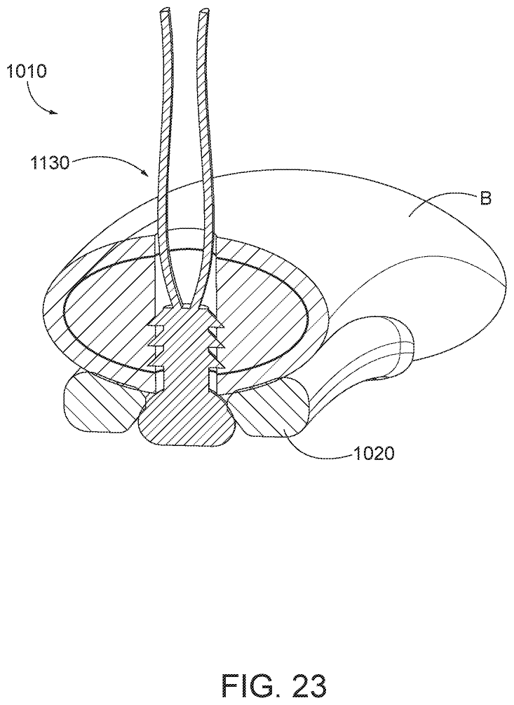

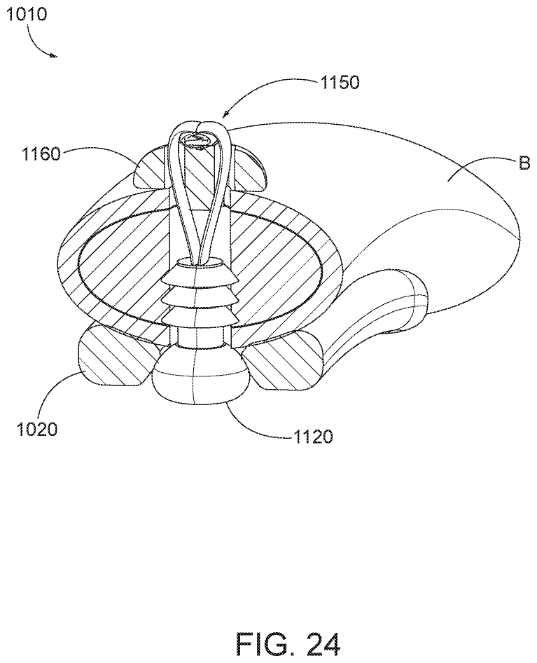

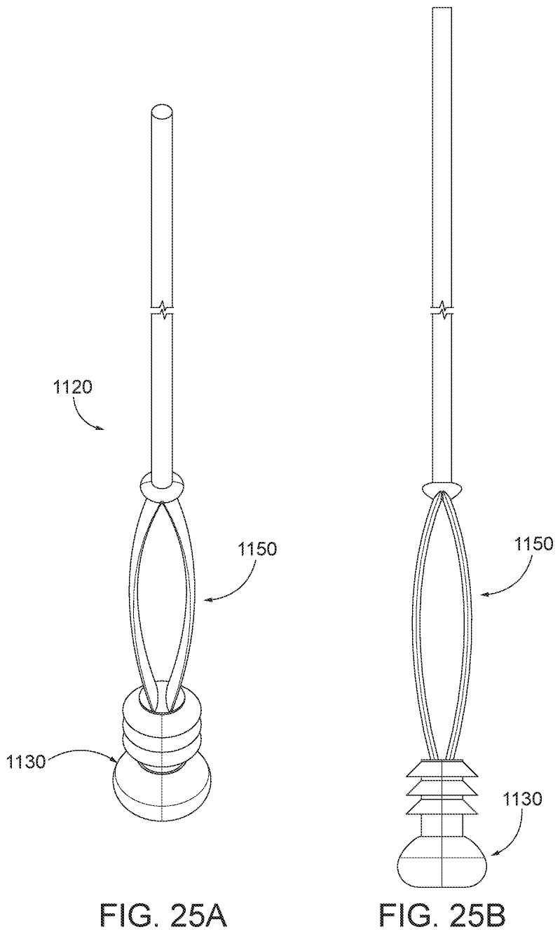

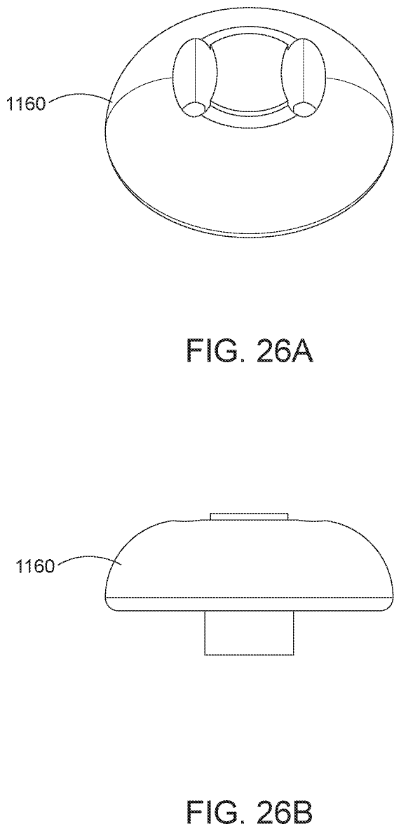

Another embodiment of a bone fixation system 1010 is illustrated in FIGS. 22-26B. The system 1010 includes a bone plate 1020 having at least one opening that extends through the plate. The system 1010 includes and at least one tethered fastener assembly 1120 configured to extend through the at least one opening. The least one tethered fastener assembly 1120 includes a fastener 1130 having a head and a suture assembly 1150 that extends relative to the head. The suture assembly 1150 includes pair of suture tails. In accordance with the another embodiment, the fastener 1130 includes a shaft that extends from the head along a central fastener axis. The suture assembly 1150 is attached to and extends from the shaft as opposed to extending directly from the head. The system 1010 also includes a locking cap 1160 having an opening sized to permit the pair of suture tails extend therethrough. The suture tails are configured to secure the locking cap 1160 in place with respect to the bone plate 1020. The sutures may be a filament or braided suture. In addition, the sutures may be bioabsorbable.

The fastener 1130 may have a number of different configurations, similar to other fasteners described in the present disclosure. For instance, the shaft may include 1) a distal portion adjacent the head and having at least one engagement feature, and 2) a proximal portion adjacent to the distal portion such that the distal portion extends from the head to the proximal portion. In such a configuration, the locking cap is configured to engage the proximal portion of the shaft but not the distal portion of the shaft. In this example, the least one engagement feature is one of: a) a keel configured to engage bone; b) a plurality of keels, c) a plurality of discrete projections, or d) a plurality of ratchet teeth. The proximal portion may define external threads and the locking cap is configured to threadably engage the external threads on the proximal portion of the shaft. In another example, the proximal portion defines external ratchet teeth and the locking cap includes internal ratchet teeth that are configured to interlock with the external ratchet teeth. The system 1010 may include an anti-rotation element on the bone plate, the fastener, or the bone plate and the fastener.

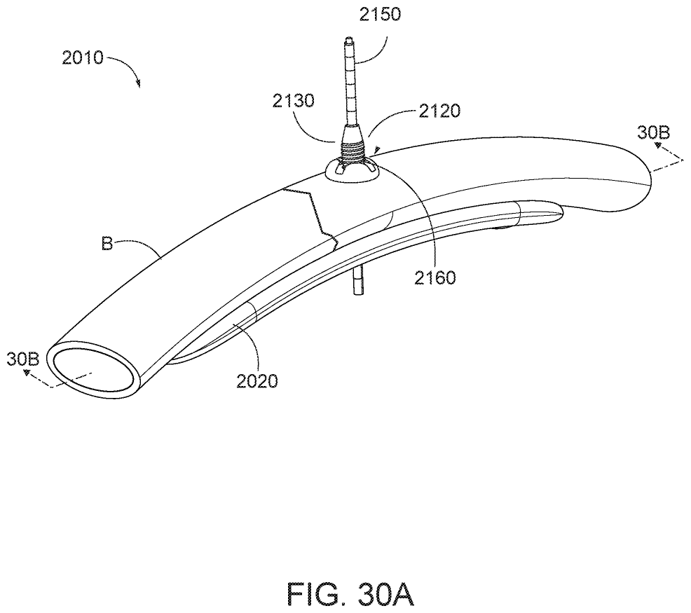

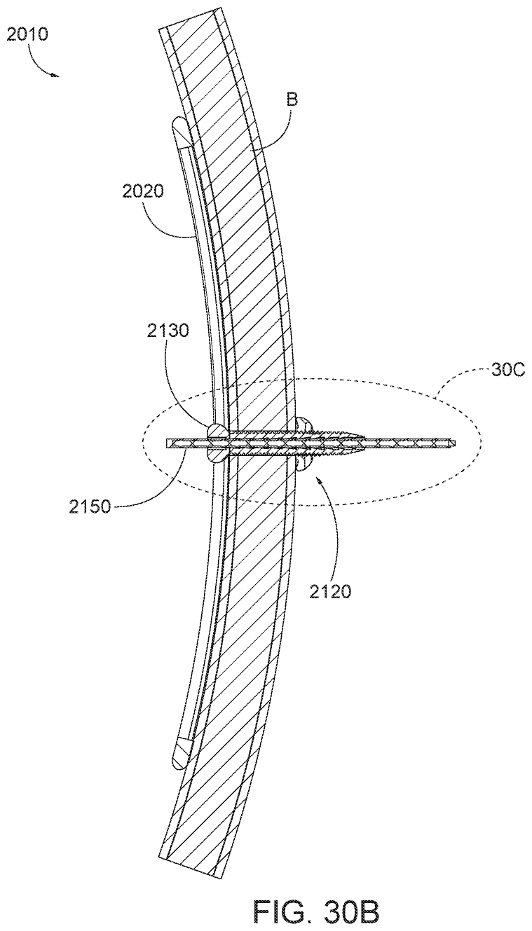

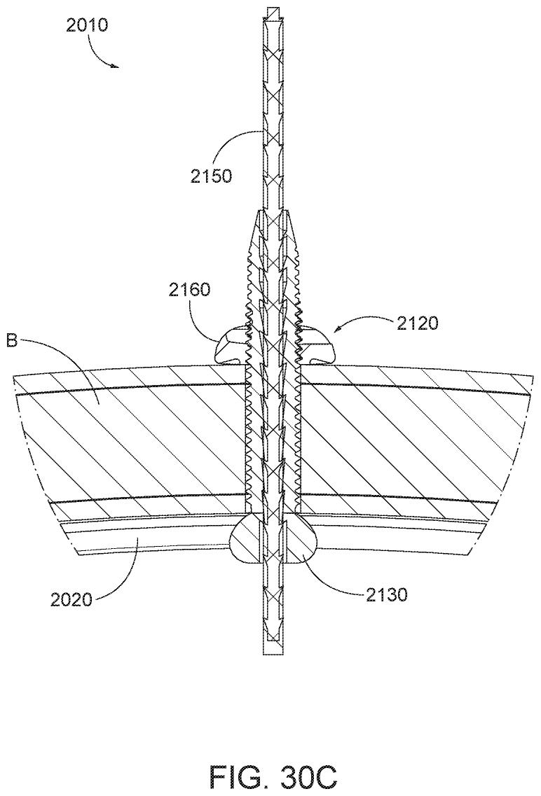

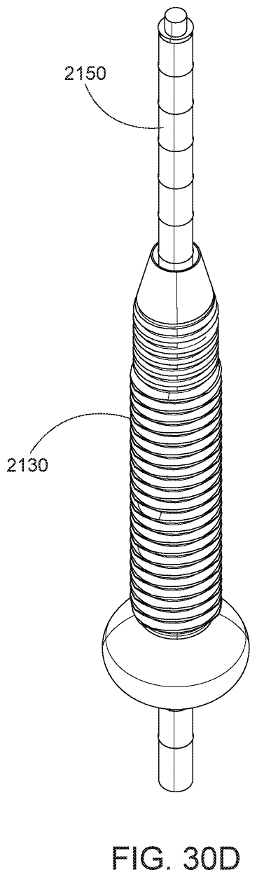

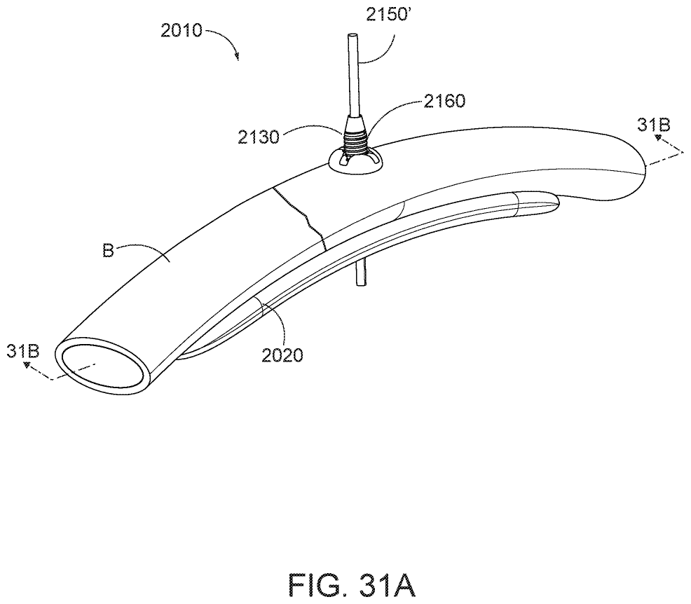

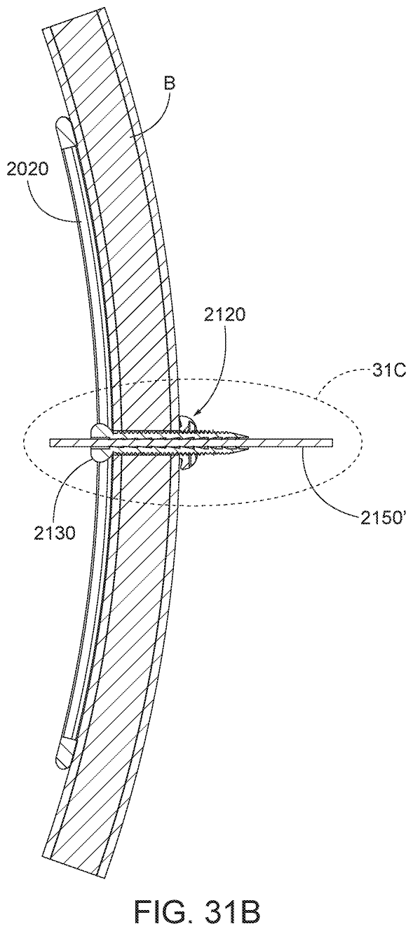

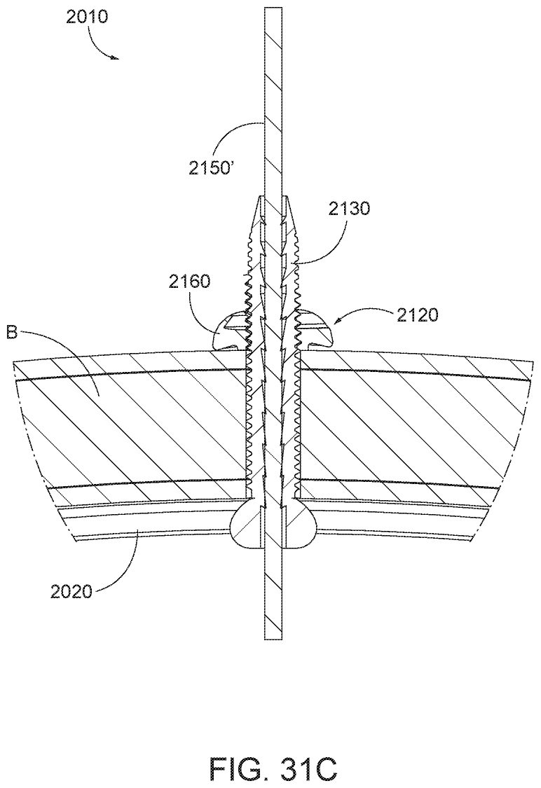

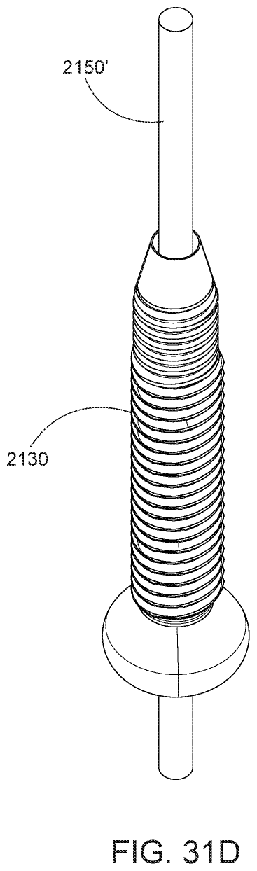

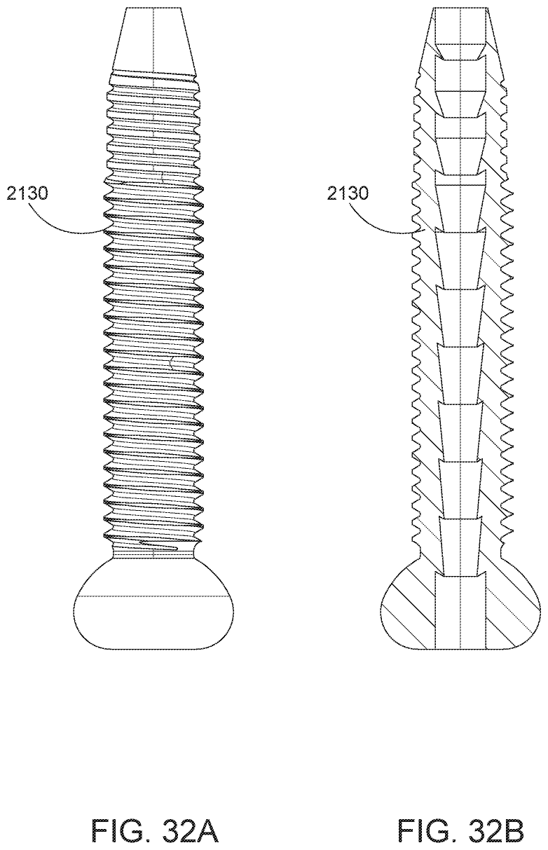

FIG. 30A-32B illustrate embodiments whereby the fastener assemblies are configured so that the flexible elongated element is movable through the fastener in only one direction. As shown in FIGS. 30A-30C, 32A, and 32B, an embodiment of the present disclosure includes a bone fixation and repair system 2010 having a bone plate 2020 configured to be placed. against a bone. The bone plate 2020 includes at least one opening that extends through the plate. The system 2010 also includes an elongated flexible element having a proximal end, a distal end, and external ratchet teeth. The system 2010 includes a plurality of a fastener assemblies 2120. The fastener assembly 2120 has a fastener 2130 has a head, a shaft that extends from the head along a central fastener axis, an inner surface, an outer surface spaced from the inner surface in a radial direction that is perpendicular to the central fastener axis, an internal channel defined by the inner surface, and an internal ratchet tooth defined by the inner surface. The internal channel is sized to received the elongated flexible element 2150. The system 2010 also includes a locking cap 2160 configured to engage the outer surface of the shaft. In another example, the internal ratchet teeth of the fastener 2130 are configured to interlock with the external ratchet teeth of the elongated flexible element 2150 such that the elongated flexible element 2150 is movable through in the internal channel in only one direction. In an alternative embodiment as shown in FIGS. 31A-31C, the flexible elongated element 2150′ has a smooth outer surface. All other respects the system shown in FIGS. 31A-31D are the same as the system shown in FIGS. 30A-30D.

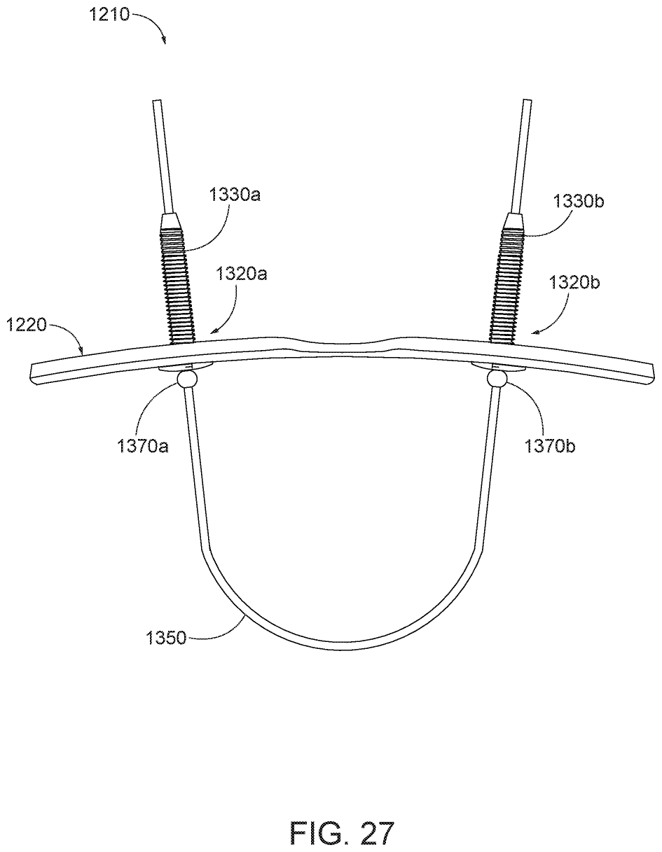

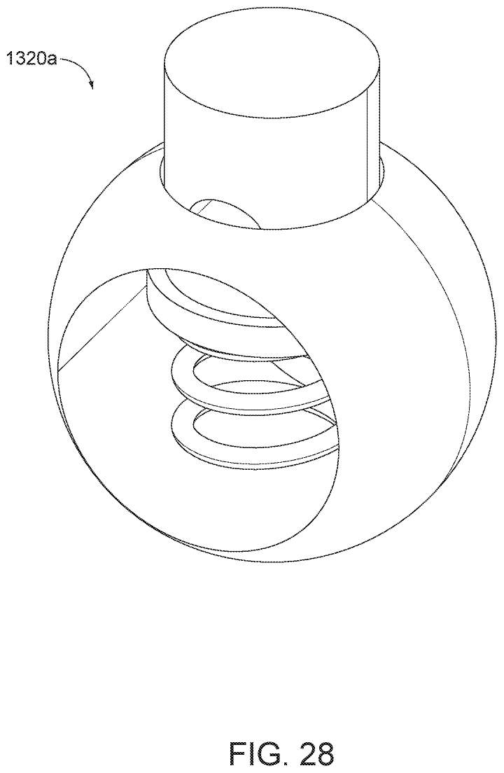

FIG. 27 illustrates another embodiment of a bone fixation and repair system 1210. As shown, the system 1210 includes bone plate 1220 having a first opening and a second opening. The system 1210 also includes a first fastener assembly 1320a disposed in the first opening. The first fastener assembly 1320a includes a first fastener 1330a that defines a first internal channel, and a first locking cap 1360a configured to be coupled to the first fastener 1330a. The system 1210 also includes a second fastener assembly 1320b disposed in the second opening. The second fastener assembly 1320b includes a second fastener 1330b that defines a second internal channel, and a second locking cap 1360b configured to be coupled to the second fastener 1330b. The first locking cap 1360a and second locking cap 1360b are not shown. The system 1210 also includes a single elongated flexible element 1350 having a proximal end, a first movable toggle 1370a associated with the first fastener assembly 1330a, and a second movable toggle 1370b associated with the second fastener assembly 1330b. The toggles 1370a, 1370b are independently movable along the elongated flexible element 1350 and configured to be fixed in position along the elongated flexible element 1350. In use, tension applied the proximal and distal ends of the elongated flexible element 1350 prevents the bone plate 1220 from sliding along the elongated flexible element 1350 when the toggles are fixed in the position and are in contact with the bone plate 1220.

In one example of the embodiment shown in FIG. 27, the first and second fasteners are threaded and the first and second locking caps are configured to threadably engage the first and second fasteners. In another example, the first and second fasteners each include external ratchet teeth, and the first and second locking caps include internal ratchet teeth that are configured to interlock the external ratchet teeth of the first and second fasteners, respectively. In the illustrated embodiment, the single elongated flexible element may be a cable, a wire, or a suture assembly.

An embodiment of the present disclosure includes a method for fixation and repair of a fracture in a rib having a first bone segment and second bone segment on either side of the fracture. The method may use the fixation system 1210 described above and shown in FIG. 27A. The method includes cutting a first and second superior incision in a thoracic cavity of a patient at a location aligned with the rib including the fracture. The method also includes cutting a third inferior incision in the thoracic cavity of the patient at a location inferior to the fracture in the rib.

The method includes drilling through the first superior incision a first hole in the first bone segment of the rib. The method includes drilling through the second superior incision a second hole in the second bone segment of the rib.

The method includes inserting a bone plate movably coupled to a single elongated flexible element through the third inferior incision to a location proximate the fracture in the rib. The user may then inert a first end of the single elongated flexible element through the first hole in the first bone segment and out through the first superior incision in a posterior-anterior direction relative to the rib. The user may also insert a second end of the single elongated flexible element through the first hole in the second bone segment and out through the second superior incision in the posterior-anterior direction relative to the rib.

The method also includes pulling the first and second ends of the single elongated flexible element in the posterior-anterior direction to 1) place the bone plate adjacent an anterior side of the rib across the fracture, 2) position the first and second fasteners through the first and second holes, respectively. The method also includes securing a first and second locking caps on the first and second fasteners, respectively.

The method also includes pulling the single elongate flexible element in an anterior-posterior direction that is opposite the posterior-anterior direction to remove the elongated flexible element from the first and second fastener assemblies.

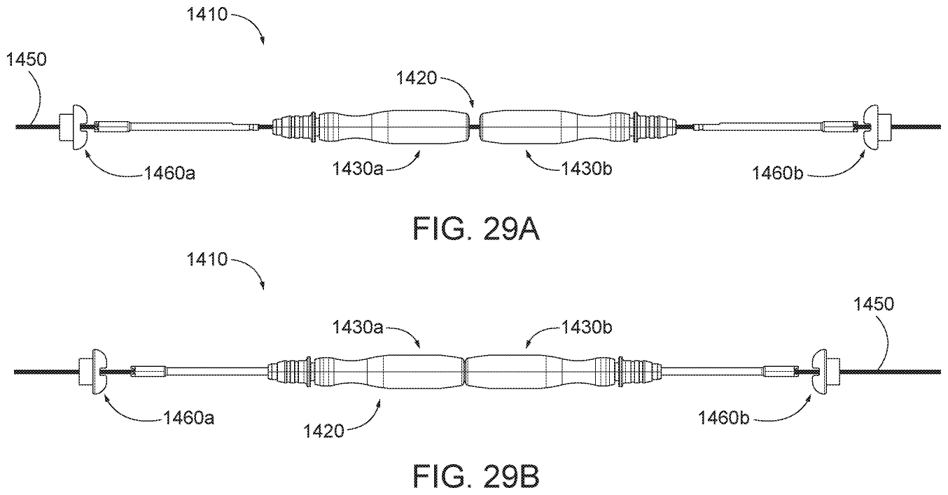

FIGS. 29A and 29B illustrate another embodiment of a bone fixation and repair system 1410 includes driving assembly 1420 integrated onto elongated flexible element 1450. In accordance with the illustrated embodiment, the system includes a bone plate having a first opening and a second opening. The bone plate is not shown in FIGS. 29A and 29B. The bone plate may be configured like any other bone plate as described in the present disclosure. For instance, the bone plate may have two openings, a single elongate slot, or a several elongated slots.

Continuing with FIGS. 29A and 29B, the system includes a first fastener assembly disposed in the first opening of the bone plate. The first fastener assembly includes a first fastener defining a first internal channel, and a first locking cap 1460a configured to be coupled to the first fastener. The system also includes a second fastener assembly disposed in the second opening of the bone plate. The second fastener assembly includes a second fastener defining a second internal channel, and a second locking cap 1460b configured to be coupled to the second fastener.

Continuing with FIGS. 29A and 29B, the system further includes a drive assembly 1420. The drive assembly 1420 may be a single drive instrument or it may comprise first and second driving instruments 1430a, and 1430b, as illustrated. As shown, the drive assembly 1420 has a first driving end, a second driving end spaced from the first driving end, and an internal channel that extends from the first driving end to the second driving end. The first driving end includes a first engagement feature configure to mate with the first locking cap 1460a, the second driving end includes a second engagement feature configure to mate with the second locking cap 1460b. The system includes a single elongated flexible element 1450 having a first terminal end, and a second terminal end. The elongated flexible element extends through the internal channel and out of the first driving end and the second driving end of the drive assembly 1420 such that the drive assembly 1420 is movable along the single elongated flexible element 1450 between the first and second terminal ends. The first and second terminal ends are configured to pass through 1) first and second holes in the bone, respectively, and 2) at least one opening in the bone plate.

In accordance with the embodiment shown in FIGS. 29A and 29B, the drive assembly 1420 comprises a first driving instrument 1430a and a second driving instrument 1430b. The first driving instrument 1430a includes the first driving end and a first proximal end opposite the first driving end. The system may also include a second driving instrument. The second driving instrument 1430b includes the second driving end and a second proximal end opposite the second driving end. In the illustrated embodiment, the first driving instrument 1430a and the second driving instrument 1430b are independently movable along the elongated flexible element 1450 to engage the first locking cap 1460a and second locking cap 1460b, respectively.

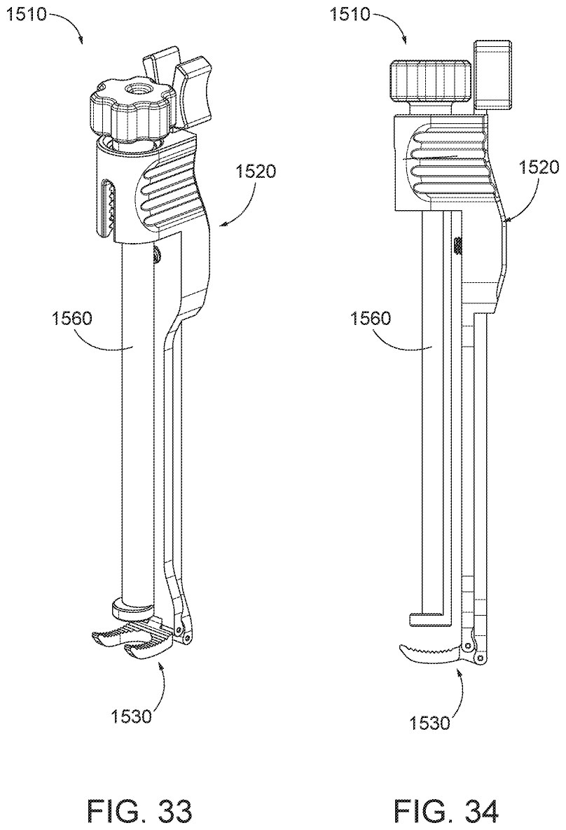

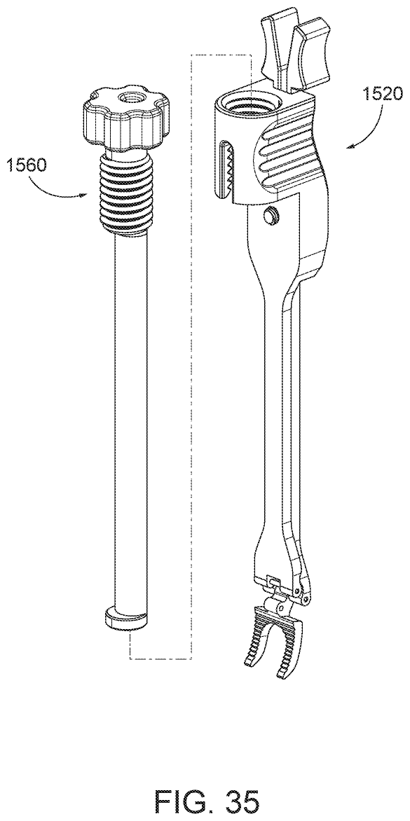

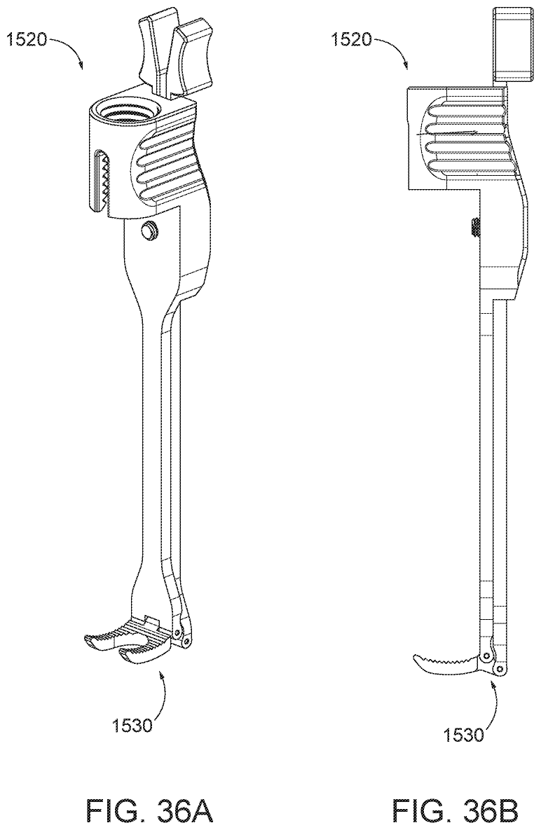

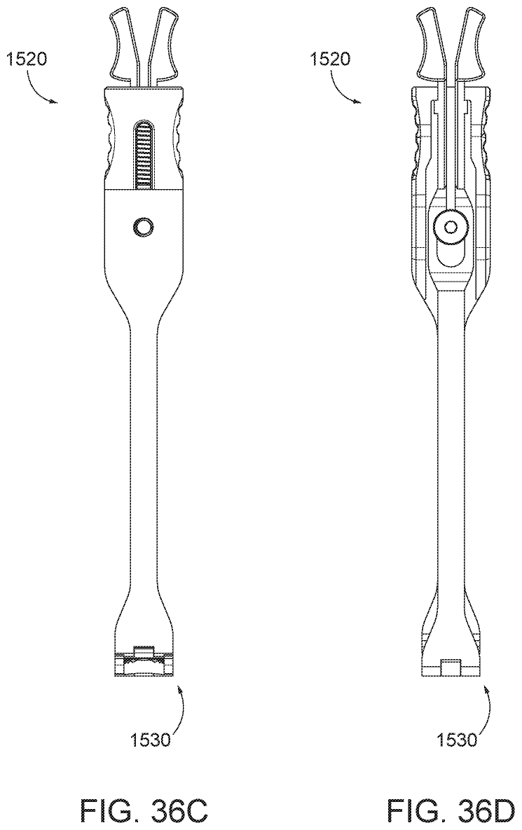

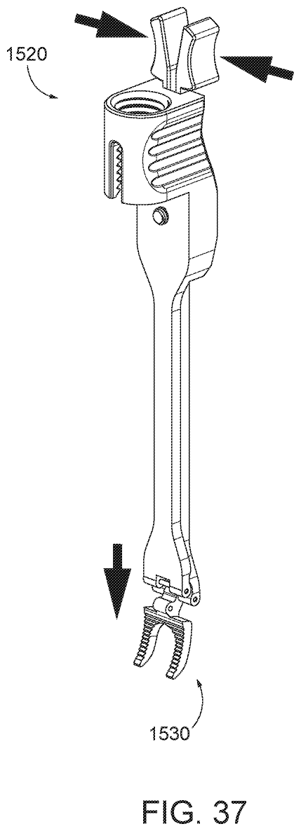

As shown in FIGS. 33-37, an embodiment of the present disclosure includes a bone fixation and repair system 1510 and instruments to facilitate implantation of the bone plate, fasteners, and locking caps as described herein. The system 1510 may include a clamping device 1520 that is elongated along a longitudinal direction. The clamping device 1520 has an articulating end includes a movable holder 1530 configured to transition between an insertion configuration, where the movable holder aligned with the longitudinal direction, and articulated configuration, where the movable holder is offset with respect to the longitudinal direction. The clamping device 1520 also includes an actuation end spaced from the articulating end along the longitudinal direction. The actuation end includes a support member with a channel and an actuator configured to transition the movable holder 1530 between the insertion configuration and the articulated configuration. Furthermore, the support member is threaded along the channel and a proximal end of the drill guide 1560 is threaded to threadably engage the support member. In addition, the clamping device 1520 includes a longitudinal member and a slide member that slidable relative to the longitudinal member. As shown, the movable holder 1530 is pivotably coupled to the longitudinal member and to the slidable member. The actuator further includes a trigger configured to permit the slide member to move along the longitudinal member, thereby causing the movable holder to pivot into the articulated configuration.

The system further includes a drill guide 1560 sized to fit in the channel, the drill guide having a distal end, a proximal end, and a lumen that extends from the distal end to the proximal end. The drill guide 1560 is configured so that when 1) the drill guide is fully seated in the support member of the clamping device, and 2) the movable holder 1530 is in the articulated configuration, the distal end of the drill guide and the movable holder define a space sized to hold the bone therein. The system may further comprise a drill configured to fit within the drill guide 1560 and drill a hole in the bone clamped by the movable holder of the clamping device 1520.

The system may be used with any bone plate and fastener assembly described in this disclosure. For instance, the system includes a bone plate having at least one opening, at least one elongated flexible element, at least one fastener assembly configured to fit within the at least one opening of the bone plate. As with other embodiments, the fastener assembly has a) a fastener and b) a locking cap configured to engage the shaft of the fastener. The fastener may include a channel that receives the at least one elongated flexible element.

An embodiment of the present disclosure includes a bone fixation and repair system, comprising a bone plate having at least one opening. The system includes at least one elongated flexible element and a fastener assembly configured to fit within the at least one opening. The fastener assembly has a) a fastener and b) a locking cap configured to engage the shaft of the fastener. In this embodiment, at least one of 1) the fastener, 2) the at least one elongated flexible element, and 3) the locking cap are bioabsorbable.

### VARIOUS NOTES AND EXAMPLES

To better illustrate the systems and methods disclosed herein, a non-limiting list of examples is provided here:

In Example 1, a bone fixation and repair system for a bone can be provided that includes a bone plate elongated along a central longitudinal axis, the bone plate having a first end, a second end spaced from the first end along the central longitudinal axis, a first and second side member on opposite sides of the central longitudinal axis, and an elongated slot that extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

In Example 2, the bone fixation and repair system of Example 1 can optionally be configured such that the internal engagement surfaces are curved.

In Example 3, the bone fixation and repair system of Example 1 can optionally be configured such that the internal engagement surfaces are flat.

In Example 4, the bone fixation and repair system of any one or any combination of Examples 1-3 can optionally be configured such that the bone plate is curved along the central longitudinal axis.

In Example 5, the bone fixation and repair system of any one or any combination of Examples 1-4 can optionally be configured such that the internal engagement surfaces a) are non-orthogonal to the depth and b) are opposed to each other to partially define the elongated slot.

In Example 6, the bone fixation and repair system of any one or any combination of Examples 1-5 can optionally be configured such that the internal engagement surfaces extend along an entirety of the first side member and the second side member.

In Example 7, the bone fixation and repair system of any one or any combination of Examples 1-6 can optionally be configured such that the bone plate defines a) a length that extends from the first end to the second end, wherein the length is perpendicular to the depth, and b) a width that extends from the first side member to the second side member, wherein the width is perpendicular to the length and the depth, wherein the elongated slot extends along an entirety of the length.

In Example 8, the bone fixation and repair system of any one or any combination of Examples 1-7 can optionally be configured such that the top surface of the first side member and the second side member are curved in order to receive a curved surface of a bone segment.

In Example 9, the bone fixation and repair system of any one or any combination of Examples 1-8 can optionally be configured such that the first and second side members include first and second ridges along the top of the first and second side members, respectively.

In Example 10, the bone fixation and repair system of any one or any combination of Examples 1-9 can optionally be configured such that the elongated slot is a first elongated slot, and the bone plate further comprises a second elongated slot.

In Example 11, the bone fixation and repair system of any one or any combination of Examples 1-10 can optionally be configured such that the elongated slot is one of a plurality of elongated slots, wherein the plurality of elongated slots have a different size and orientation with respect to each other.

In Example 12, the bone fixation and repair system of Example 11 can optionally be configured such that the bone plate further comprises at least two openings that extend through the bone plate along the depth.

In Example 13, the bone fixation and repair system of any one or any combination of Examples 1-10 can optionally be configured such that the bone plate further comprises at least two openings that extend through the bone plate along the depth.

In Example 14, the bone fixation and repair system of any one or any combination of Examples 1-13 can optionally be configured such that the elongated slot is a first elongated slot, and the bone plate comprises: a first segment including the first side member, the second side member, and the first elongated slot; a second segment including a first side member, a second side member, a second elongated slot, where the first side member and the second side member of the second segment is substantially similar to the first side member and the second side member of the first segment; and a bridge segment that couples the first segment to the second segment, the bridge segment defining a bridge slot that is open the first and second elongated slots, such that a tethered fastener assembly is movable along any portion of the first elongated slot, the second elongated slot, and the bridge slot.

In Example 15, the bone fixation and repair system of any one or any combination of Examples 1-14 can optionally be configured such that system further comprises: a plurality of a tethered fastener assemblies configured to concurrently fit within and slide along any portion of the elongated slot, each tethered fastener assembly having 1) a fastener having a head, a shaft that extends from the head along a central fastener axis, and an elongated flexible element that is attached to and extends from the shaft, and 2) a locking cap configured to slide along the elongated flexible element into engagement with the shaft, wherein the head defines a distal end of the tethered fastener assembly and the elongated flexible element defines a proximal end of the assembly such that the tethered fastener assembly is an integrated unit.

In Example 16, the bone fixation and repair system of Example 15 can optionally be configured such that the head defines a smooth outer surface configured to movably engage the internal engagement surfaces of the bone plate, such that the fastener is rotatable about multiple axes when the smooth outer surface of the head is in contact the internal engagement surfaces of the bone plate.

In Example 17, the bone fixation and repair system of Example 16 can optionally be configured such that the smooth outer surface of the head is curved with respect to the central fastener axis.

In Example 18, the bone fixation and repair system of Example 16 can optionally be configured such that the smooth outer surface of the head is linear and angled with respect to the central fastener axis.

In Example 19, the bone fixation and repair system of any one or any combination of Examples 15-18 can optionally be configured such that the plurality of tethered fastener assemblies are sized and configured to concurrently fit within and slide along any portion of the elongated slot.

In Example 20, the bone fixation and repair system of any one or any combination of Examples 15-19 can optionally be configured such that the elongated slot is sized to permit the shaft to pass therethrough but inhibit the head to pass therethrough.

In Example 21, the bone fixation and repair system of any one or any combination of Examples 15-20 can optionally be configured such that the locking cap is configured to fix the fastener in the elongated slot of the bone plate when a) the bone plate is in contact with the bone, and b) the locking cap is engaged with the shaft and in contact with the bone.

In Example 22, the bone fixation and repair system of any one or any combination of Examples 15-21 can optionally be configured such that at least a portion of the shaft is threaded.

In Example 23, the bone fixation and repair system of Example 22 can optionally be configured such that the locking cap includes an internal channel and internal threads that are configured for threaded engagement with the shaft.

In Example 24, the bone fixation and repair system of any one or any combination of Examples 15-23 can optionally be configured such that the elongated flexible element is a cable.

In Example 25, the bone fixation and repair system of any one or any combination of Examples 15-23 can optionally be configured such that the elongated flexible element is a suture.

In Example 26, the bone fixation and repair system of any one or any combination of Examples 15-23 can optionally be configured such that the elongated flexible element is a wire.

In Example 27, the bone fixation and repair system of Example 15 can optionally be configured such that the shaft extends from the head to a shaft tip, wherein the shaft tip defines a tip engagement feature configured to receive a torque.

In Example 28, the bone fixation and repair system of Example 27 can optionally be configured such that the shaft is threaded.

In Example 29, the bone fixation and repair system of Example 27 or Example 28 can optionally be configured such that the system further comprises: an outer driving instrument defining a proximal end and a distal end opposite the proximal end, and an internal channel that extends from the proximal end to the distal end, the distal end defining an engagement member configured to engage an upper surface of the locking cap so as to drive the locking cap into engagement with the shaft of the fastener; and an internal driving instrument having a back end, a driving end, and an outer surface, the outer surface slidable within the internal channel of the outer driving instrument, the driving end defining an instrument engagement feature that mates with the tip engagement feature such that the internal driving instrument is configured to apply the torque to the shaft tip of the fastener when the instrument engagement feature is mated with the tip engagement feature.

In Example 30, a bone fixation and repair system for a bone can be provided that includes a bone plate and a plurality of tethered fastener assemblies. The bone plate can be configured to be placed against a bone, wherein the bone plate includes at least one opening. The plurality of tethered fastener assemblies can be configured to secure the bone plate to the bone, each tethered fastener assembly having: a fastener having a head, a shaft that extends from the head along a central fastener axis, and an elongated flexible element attached to and extending from the shaft, the shaft defining external ratchet teeth, wherein the head defines a distal end of the assembly and the elongated flexible element defines a proximal end of the assembly such that the tethered fastener assembly is an integrated unit, and a locking cap configured to be attached to the fastener, the locking cap including a top, a bottom, an inner surface, and an inner opening defined by the inner surface, the inner surface defining internal ratchet teeth, wherein the internal ratchet teeth are configured to engage the external ratchet teeth of the shaft so as to lock the locking cap to the fastener.

In Example 31, the bone fixation and repair system of Example 30 can optionally be configured such that the locking cap is configured to fix the fastener in the at least one opening of the bone plate when a) the bone plate is in contact with the bone, and b) the locking cap is engaged with the shaft and in contact with the bone.

In Example 32, the bone fixation and repair system of Example 30 or Example 31 can optionally be configured such that the shaft has 1) a distal portion adjacent the head and having at least one engagement feature, and 2) a proximal portion defining the external ratchet teeth.

In Example 33, the bone fixation and repair system of Example 32 can optionally be configured such that the at least one engagement feature is one of: a) a keel configured to engage bone; b) a plurality of keels, or c) a plurality of discrete projections that extend outwardly away from the shaft, each projection being spaced apart and separate from other projections.

In Example 34, the bone fixation and repair system of Example 33 can optionally be configured such that the shaft extends along a central axis, wherein each keel extends outwardly from the shaft along a radial direction that this perpendicular to the central axis.

In Example 35, the bone fixation and repair system of any one or any combination of Examples 30-34 can optionally be configured such that the at least one opening is an elongated slot, wherein the bone plate is elongated along a central longitudinal axis, the bone plate having a first end, a second end spaced from the first end along the central longitudinal axis, a first and second side member on opposite sides of the central longitudinal axis, and the elongated slot extends through the bone plate along a depth that is perpendicular to the central longitudinal axis, the first side member and second side member each defining a bottom surface, a top surface spaced from the bottom. surface along the depth, and an internal engagement surface that extends from the bottom surface toward the central longitudinal axis.

In Example 36, the bone fixation and repair system of Example 35 can optionally be configured such that the head defines a smooth outer surface configured to movably engage the internal engagement surface of the bone plate, such that, the fastener is rotatable about multiple axes when the smooth outer surface is in contact the internal engagement surface.

In Example 37, the bone fixation and repair system of Example 35 or Example 36 can optionally be configured such that the internal engagement surfaces a) are non-orthogonal to the depth and b) are opposed to each other to partially define the elongated slot.

In Example 38, the bone fixation and repair system of any one or any combination of Examples 35-37 can optionally be configured such that the internal engagement surfaces extend along an entirety of the first side member and the second side member.

In Example 39, the bone fixation and repair system of any one or any combination of Examples 35-38 can optionally be configured such that the bone plate is curved along the central longitudinal axis.

In Example 40, the bone fixation and repair system of any one or any combination of Examples 35-38 can optionally be configured such that the bone plate is substantially flat along the central longitudinal axis.

In Example 41, a bone fixation and repair system for a bone can be provided that includes a bone plate and at least one tethered fastener assembly. The bone plate can be configured to be placed against a bone, and the bone plate can include at least one opening. The at least one tethered fastener assembly can be configured to extend through the at least one opening, the at least one tethered fastener assembly having: a fastener having a head, a shaft that extends from the head along a central fastener axis, and an elongated flexible element attached to and extending from the shaft, the shaft having 1) a distal portion adjacent the head and having at least one engagement feature, and 2) a proximal portion adjacent to the distal portion such that the distal portion extends from the head to the proximal portion; and a locking cap configured to engage the proximal portion of the shaft but not the distal portion of the shaft.

In Example 42, the bone fixation and repair system of Example 41 can optionally be configured such that the proximal portion defines external threads, wherein the locking cap is configured to threadably engage the external threads on the proximal portion of the shaft.

In Example 43, the bone fixation and repair system of Example 41 can optionally be configured such that the proximal portion defines external ratchet teeth, wherein the locking cap includes internal ratchet teeth that are configured to interlock with the external ratchet teeth.

In Example 44, the bone fixation and repair system of any one of Examples 41, 42, or 43 can optionally be configured such that the at least one engagement feature is a keel configured to engage bone.

In Example 45, the bone fixation and repair system of Example 44 can optionally be configured such that the shaft extends along a central fastener axis, wherein the keel extends outwardly from the shaft along a radial direction that this perpendicular to the central fastener axis.

In Example 46, the bone fixation and repair system of any one of Examples 41, 42, or 43 can optionally be configured such that the at least one engagement feature is a plurality of keels.

In Example 47, the bone fixation and repair system of any one of Examples 41, 42, or 43 can optionally be configured such that the shaft extends along a central axis, wherein each keel extends outwardly from the shaft along a radial direction that this perpendicular to the central axis.

In Example 48, the bone fixation and repair system of any one of Examples 41, 42, or 43 can optionally be configured such that the at least one engagement feature is a plurality of discrete projections that extend outwardly away from the shaft, each projection being spaced apart and separate from other projections.

In Example 49, the bone fixation and repair system of any one of Examples 41, 42, or 43 can optionally be configured such that the system further comprises a locking cap configured to be attached to the fastener, the locking cap including a top, a bottom, an inner surface, and an inner opening defined by the inner surface, the inner surface defining internal threads that are configured to engage the external threads of the shaft so as to lock the locking cap to the fastener.

In Example 50, the bone fixation and repair system of any one of Examples 41, 42, or 43 can optionally be configured such that the at least one opening is an elongated slot, wherein the bone plate is elongated along a central longitudinal axis, the bone plate having a first end, a second end spaced from the first end along the central longitudinal axis, a first and second side member on opposite sides of the central longitudinal axis, and the elongated slot extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

In Example 51, the bone fixation and repair system of Example 50 can optionally be configured such that the head defines a smooth outer surface configured to movably engage the internal engagement surface of the bone plate, such that, the fastener is rotatable about multiple axes when the smooth outer surface is in contact the internal engagement surface.

In Example 52, the bone fixation and repair system of Example 50 or Example 51 can optionally be configured such that the internal engagement surfaces a) are non-orthogonal to the depth and b) are opposed to each other to partially define the elongated slot.

In Example 53, the bone fixation and repair system of Example 50 or Example 51 can optionally be configured such that the internal engagement surfaces extend along an entirety of the first side member and the second side member.

In Example 54, a bone fixation and repair system for a bone can be provided that includes a bone plate and at least one tethered fastener assembly. The bone plate can be configured to be placed against a bone, the bone plate including a top configured to face the bone, a bottom opposite the top, at least one opening that extends from the bottom to the top, and a first anti-rotation element disposed along the bottom adjacent the at least one opening. The at least one tethered fastener assembly can be configured to extend through the at least one opening, the at least one tethered fastener assembly having a fastener having a head, a shaft that extends from the head along a central fastener axis, and an elongated flexible element attached to and extending from the shaft, the head defining a distal end of the assembly and the elongated flexible element defining a proximal end of the assembly such that the tethered fastener assembly is an integrated unit, the head having a second anti-rotation element configured to engage the first anti-rotation element such that when the fastener is fully seated in the at least one opening, wherein the first anti-rotation element and the second anti-rotation element interlock so that the fastener is inhibited from rotation with respect to the bone plate, and a locking cap configured to engage the shaft.

In Example 55, the bone fixation and repair system of Example 54 can optionally be configured such that the first anti-rotation element is a plurality of spaced apart grooves disposed along the bottom of the bone plate, and the second anti-rotation element is plurality of linear ridges that extend outwardly from the head from a proximal surface of the head toward the shaft, wherein each projection is sized to engage each groove.

In Example 56, the bone fixation and repair system of Example 55 can optionally be configured such that the second anti-rotation element is plurality of discrete projections that extend outwardly from the head, wherein one or more of the plurality of discrete projections are configured to engage each one of the grooves.

In Example 57, the bone fixation and repair system of any one or any combination of Examples 54-56 can optionally be configured such that the shaft is threaded and the locking cap is configured to threadably engage the shaft.

In Example 58, the bone fixation and repair system of any one or any combination of Examples 54-56 can optionally be configured such that the shaft defines external ratchet teeth and the locking cap defines internal ratchet teeth configured to interlock with the external ratchet teeth.

In Example 59, the bone fixation and repair system of any one or any combination of Examples 54-58 can optionally be configured such that the elongated flexible element is a suture assembly, the suture assembly include a pair suture tails that configured to secure the locking cap in place with respect to the head of the tethered fastener assembly.

In Example 60, the bone fixation and repair system of any one or any combination of Examples 54-59 can optionally be configured such that the at least one opening is an elongated slot, wherein the bone plate is elongated along a central longitudinal axis, the bone plate having a first end, a second end spaced from the first end along the central longitudinal axis, a first and second side member on opposite sides of the central longitudinal axis, and the elongated slot extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

In Example 61, the bone fixation and repair system of Example 60 can optionally be configured such that the head defines a smooth outer surface configured to movably engage the internal engagement surface of the bone plate, such that, the fastener is rotatable about multiple axes when the smooth outer surface is in contact the internal engagement surface.

In Example 62, the bone fixation and repair system of Example 60 or Example 61 can optionally be configured such that the bone plate is curved along the central longitudinal axis.

In Example 63, the bone fixation and repair system of Example 60 or Example 61 can optionally be configured such that the bone plate is substantially flat along the central longitudinal axis.

In Example 64, the bone fixation and repair system of any one or any combination of Examples 60-63 can optionally be configured such that the internal engagement surfaces as are non-orthogonal to the depth and b) are opposed to each other to partially define the elongated slot.

In Example 65, the bone fixation and repair system of any one or any combination of Examples 60-64 can optionally be configured such that the internal engagement surfaces extend along an entirety of the first side member and the second side member.

In Example 66, a bone fixation and repair system for a bone can be provided that includes a bone plate and at least one tethered fastener assembly. The bone plate can be configured to be placed against a bone, the bone plate including a top configured to face the bone, a bottom opposite the top, at least one opening that extends from the bottom to the top, and an anti-rotation element disposed along the bottom adjacent the at least one opening. The at least one tethered fastener assembly can be configured to extend through the at least one opening, the at least one tethered fastener assembly having a fastener having a head, a shaft that extends from the head along a central fastener axis, and an elongated flexible element attached to and extending from the shaft, wherein when the fastener is fully seated in the at least one opening, the head and the anti-rotation elements engage so that the fastener is inhibited from rotation with respect to the bone plate, and a locking cap configured to engage the shaft.

In Example 67, the bone fixation and repair system of Example 66 can optionally be configured such that the anti-rotation element is a plurality of spaced apart grooves disposed along the bottom of the bone plate.

In Example 68, the bone fixation and repair system of Example 66 can optionally be configured such that the anti-rotation element is a plurality of spaced apart projections disposed along the bottom of the bone plate.

In Example 69, a bone fixation and repair system for a bone can be provided that includes a bone plate and at least one tethered fastener assembly. The bone plate can be configured to be placed against a bone, the bone plate including a top configured to face the bone, a bottom opposite the top, and at least one opening that extends from the bottom to the top. The at least one tethered fastener assembly can be configured to extend through the at least one opening, the at least one tethered fastener assembly having a fastener having a head, a shaft that extends from the head along a central fastener axis, and an elongated flexible element attached to and extending from the shaft, the head having an anti-rotation element configured to engage the bottom of the bone plate such that when the fastener is fully seated in the at least one opening, the anti-rotation element and the bottom of the bone plate interlock so that the fastener is inhibited from rotation with respect to the bone plate, and a locking cap configured to engage the shaft.

In Example 70, the bone fixation and repair system of Example 69 can optionally be configured such that the anti-rotation element is a plurality of discrete projections that extend outwardly from the head.

In Example 71, the bone fixation and repair system of Example 69 can optionally be configured such that the anti-rotation element is plurality of discrete ridges that extend outwardly from the head.

In Example 72, the bone fixation and repair system of Example 69 or Example 70 can optionally be configured such that the shaft is threaded and the locking cap is configured to threadably engage the shaft.

In Example 73, the bone fixation and repair system of Example 69 or Example 70 can optionally be configured such that the shaft defines external ratchet teeth and the locking cap defines internal ratchet teeth configured to interlock with the external ratchet teeth.

In Example 74, the bone fixation and repair system of any one or any combination of Examples 69-73 can optionally be configured such that the elongated flexible element is a suture assembly, the suture assembly include a pair suture tails that configured to secure the locking cap in place with respect to the head of the tethered fastener assembly.

In Example 75, the bone fixation and repair system of any one or any combination of Examples 69-74 can optionally be configured such that the at least one opening is an elongated slot, wherein the bone plate is elongated along a central longitudinal axis, the bone plate having a first end, a second end spaced from the first end along the central longitudinal axis, a first and second side member on opposite sides of the central longitudinal axis, and the elongated slot extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

In Example 76, the bone fixation and repair system of Example 75 can optionally be configured such that the head defines a smooth outer surface configured to movably engage the internal engagement surface of the bone plate, such that, the fastener is rotatable about multiple axes when the smooth outer surface is in contact the internal engagement surface.

In Example 77, the bone fixation and repair system of any one or any combination of Examples 69-76 can optionally be configured such that the bone plate is curved along the central longitudinal axis.

In Example 78, the bone fixation and repair system of any one or any combination of Examples 69-76 can optionally be configured such that the bone plate is substantially fiat along the central longitudinal axis.

In Example 79, the bone fixation and repair system of any one or any combination of Examples 75-78 can optionally be configured such that the internal engagement surfaces a) are non-orthogonal to the depth and b) are opposed to each other to partially define the elongated slot.

In Example 80, the bone fixation and repair system of any one or any combination of Examples 75-79 can optionally be configured such that the internal engagement surfaces extend along an entirety of the first side member and the second side member.

In Example 81, a bone fixation and repair system for a bone can be provided that includes a bone plate and at least one tethered fastener assembly. The bone plate can be configured to be placed against a bone, the bone plate including at least one opening. The at least one tethered fastener assembly can be configured to extend through the at least one opening, the at least one tethered fastener assembly having: a fastener having a head and a suture assembly that extends relative to the head, the suture assembly including a pair of suture tails; and a locking cap having an opening sized to permit the pair of suture tails extend therethrough, wherein the pair of suture tails are configured to secure the locking cap in place with respect to the bone plate.

In Example 82, the bone fixation and repair system of Example 81 can optionally be configured such that the fastener includes a shaft that extends from the head along a central fastener axis, and the suture assembly is attached to and extends from the shaft.

In Example 83, the bone fixation and repair system of Example 82 can optionally be configured such that the shaft has 1) a distal portion adjacent the head and having at least one engagement feature, and 2) a proximal portion adjacent to the distal portion such that the distal portion extends from the head to the proximal portion, wherein the locking cap configured to engage the proximal portion of the shaft but not the distal portion of the shaft.

In Example 84, the bone fixation and repair system of Example 83 can optionally be configured such that the proximal portion defines external threads, wherein the locking cap is configured to threadably engage the external threads on the proximal portion of the shaft.

In Example 85, the bone fixation and repair system of Example 83 can optionally be configured such that the proximal portion defines external ratchet teeth, wherein the locking cap include internal ratchet teeth that are configured to interlock with the external ratchet teeth.

In Example 86, the bone fixation and repair system of any one of Examples 83-85 can optionally be configured such that the at least one engagement feature is one of a) a keel configured to engage bone; b) a plurality of keels, or c) a plurality of discrete projections that extend outwardly away from the shaft, each projection being spaced apart and separate from other projections.

In Example 87, the bone fixation and repair system of Example 86 can optionally be configured such that the shaft extends along a central axis, wherein each keel extends outwardly from the shaft along a radial direction that this perpendicular to the central axis.

In Example 88, the bone fixation and repair system of any one or any combination of Examples 81-87 can optionally be configured such that the suture tails comprise a filament or braided suture.

In Example 89, the bone fixation and repair system of any one or any combination of Examples 81-87 can optionally be configured such that the at least one of the a) bone plate, b) fastener assembly, and c) suture assembly is bioabsorbable.

In Example 90, the bone fixation and repair system of any one or any combination of Examples 81-89 can optionally be configured such that the at least one opening is an elongated slot, wherein the bone plate is elongated along a central longitudinal axis, the bone plate having a first end, a second end spaced from the first end along the central longitudinal axis, a first and second side member on opposite sides of the central longitudinal axis, and the elongated slot extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

In Example 91, the bone fixation and repair system of Example 90 can optionally be configured such that the head defines a smooth outer surface configured to movably engage the internal engagement surface of the bone plate, such that, the fastener is rotatable about multiple axes when the smooth outer surface is in contact the internal engagement surface.

In Example 92, the bone fixation and repair system of Example 90 or Example 91 can optionally be configured such that the bone plate is curved along the central longitudinal axis.

In Example 93, the bone fixation and repair system of Example 90 or Example 91 can optionally be configured such that the bone plate is substantially flat along the central longitudinal axis.

In Example 94, the bone fixation and repair system of any one or any combination of Examples 90-93 can optionally be configured such that the internal engagement surfaces a) are non-orthogonal to the depth and b) are opposed to each other to partially define the elongated slot.

In Example 95, the bone fixation and repair system of any one or any combination of Examples 90-94 can optionally be configured such that the internal engagement surfaces extend along an entirety of the first side member and the second side member.

In Example 96, the bone fixation and repair system of any one or any combination of Examples 81-95 can optionally be configured such that the bone plate includes an anti-rotation element.

In Example 97, the bone fixation and repair system of any one or any combination of Examples 81-96 can optionally be configured such that the fastener includes an anti-rotation element.

In Example 98, a bone fixation and repair system for a bone can include a bone plate, an elongated flexible element, and a plurality of fastener assemblies. The bone plate can be configured to be placed against a bone, the bone plate including at least one opening. The elongated flexible element can have a proximal end, a distal end, and external ratchet teeth. Each of the plurality of fastener assemblies can have: a fastener having a head, a shaft that extends from the head along a central fastener axis, an inner surface an outer surface spaced from the inner surface in a radial direction that is perpendicular to the central fastener axis, an internal channel defined by the inner surface, and an internal ratchet teeth defined by the inner surface, wherein the internal channel is sized to receive the elongated flexible element, and a locking cap configured to engage the outer surface of the shaft, wherein the internal ratchet teeth of the fastener are configured to interlock with the external ratchet teeth of the elongated flexible element such that the elongated flexible element is movable through in the internal channel in only one direction.

In Example 99, the bone fixation and repair system of Example 98 can optionally be configured such that the head defines a smooth outer surface configured to movably engage an internal engagement surface of the bone plate, such that, the fastener is rotatable about multiple axes when the smooth outer surface is in contact the internal engagement surface.

In Example 100, the bone fixation and repair system of Example 98 or Example 99 can optionally be configured such that the locking cap is configured to secure the fastener in the at least one opening of the bone plate when a) the bone plate is in contact with the bone, and b) the locking cap is engaged with the shaft and in contact with the bone.

In Example 101, the bone fixation and repair system of any one or any combination of Examples 98-100 can optionally be configured such that the at least one opening is an elongated slot, wherein the bone plate is elongated along a central longitudinal axis, the bone plate having a first end, a second end spaced from the first end along the central longitudinal axis, a first and second side member on opposite sides of the central longitudinal axis, and the elongated slot extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

In Example 102, the bone fixation and repair system of Example 101 can optionally be configured such that the head defines a smooth outer surface configured to movably engage the internal engagement surface of the bone plate, such that, the fastener is rotatable about multiple axes when the smooth outer surface is in contact the internal engagement surface.

In Example 103, the bone fixation and repair system of Example 101 or Example 102 can optionally be configured such that the bone plate is curved along the central longitudinal axis.

In Example 104, the bone fixation and repair system of Example 101 or Example 102 can optionally be configured such that the bone plate is substantially flat along the central longitudinal axis.

In Example 105, the bone fixation and repair system of any one or any combination of Examples 101-104 can optionally be configured such that the internal engagement surfaces a) are non-orthogonal to the depth and b) are opposed to each other to partially define the elongated slot.

In Example 106, the bone fixation and repair system of any one or any combination of Examples 101-105 can optionally be configured such that the internal engagement surfaces extend along an entirety of the first side member and the second side member.

In Example 107, a bone fixation and repair system can be provided that includes a bone plate, a first fastener assembly, a second fastener assembly, and a single elongated flexible element. The bone plate can have a first opening and a second opening. The first fastener assembly can be disposed in the first opening, the first fastener assembly including a first fastener defining a first internal channel, and a first locking cap configured to be coupled to the first fastener. The second fastener assembly can be disposed in the second opening, the second fastener assembly including a second fastener defining a second internal channel, and a second locking cap configured to be coupled to the second fastener. The single elongated flexible element can have a proximal end, a first movable toggle associated with the first fastener assembly, and a second movable toggle associated with the second fastener assembly, each toggle being independently movable along the single elongated flexible element and configured to be fixed in position along the elongated flexible element, wherein tension applied the proximal end and the distal end of the elongated flexible element prevent the bone plate from sliding along the elongated flexible element when the toggles are fixed in position and are in contact with the bone plate.

In Example 108, a bone fixation and repair system can be provided that includes a bone plate, a first fastener assembly, a second fastener assembly, a drive assembly, and a single elongated flexible element. The bone plate can have a first opening and a second opening. The first fastener assembly can be disposed in the first opening, the first fastener assembly including a first fastener defining a first internal channel, and a first locking cap configured to be coupled to the first fastener. The second fastener assembly can be disposed in the second opening, the second fastener assembly including a second fastener defining a second internal channel, and a second locking cap configured to be coupled to the second fastener. The drive assembly can have a first driving end, a second driving end spaced from the first driving end, and an internal channel that extends from the first driving end to the second driving end, the first driving end including a first engagement feature configure to mate with the first locking cap, the second driving end including a second engagement feature configure to mate with the second locking cap. The single elongated flexible element can have a first terminal end, and a second terminal end, wherein the single elongated flexible element extends through the internal channel and out of the first driving end and the second driving end of the drive assembly, such that the drive assembly is movable along the single elongated flexible element between the first and second terminal ends.

In Example 109, the bone fixation and repair system of Example 108 can optionally be configured such that the drive assembly comprises: a first driving instrument, the first driving instrument including the first driving end and a first proximal end opposite the first driving end; and a second driving instrument, the second driving instrument including the second driving end and a second proximal end opposite the second driving end, wherein the first and second driving instruments are independently movable along the single elongated flexible element to engage the first and second locking caps respectively, wherein the first proximal end and the second proximal end are positioned to face each other along the single elongated flexible element.

In Example 110, the bone fixation and repair system of Example 108 or Example 109 can optionally be configured such that the elongated flexible element comprises a first movable toggle associated with the first fastener assembly and a second movable toggle associated with the second fastener assembly, each toggle being independently movable along the elongated flexible element and configured to be fixed in position along the single elongated flexible element.

In Example 111, the bone fixation and repair system of Example 110 can optionally be configured such that the bone plate is positioned between the first and second movable toggles and the drive assembly.

In Example 112, the bone fixation and repair system of Example 110 or Example 111 can optionally be configured such that tension applied to the terminal ends of the elongated flexible element prevent the bone plate from sliding along the single elongated flexible element when the toggles are fixed in position and are in contact with the bone plate.

In Example 113, the bone fixation and repair system of any one or any combination of Examples 108-112 can optionally be configured such that the first and second fasteners are threaded and the first and second locking caps are configured to threadably engage the first and second fasteners.

In Example 114, the bone fixation and repair system of any one or any combination of Examples 108-112 can optionally be configured such that the first and second fasteners each include external ratchet teeth, and the first and second locking caps each include internal ratchet teeth that are configured to interlock the external ratchet teeth of the first and second fasteners, respectively.

In Example 115, the bone fixation and repair system of any one or any combination of Examples 108-114 can optionally be configured such that the single elongated flexible element is cable, a wire, or a suture assembly.

In Example 116, the bone fixation and repair system of any one or any combination of Examples 108-115 can optionally be configured such that the first and second terminal ends are configured to pass through 1) first and second holes in a bone, respectively, and 2) at least one opening in the bone plate.

In Example 117, a method for fixation and repair of a fracture in a rib having a first bone segment and second bone segment on either side of the fracture can include: cutting a first and second superior incision in a thoracic cavity of a patient at a location aligned with the rib including the fracture; cutting a third inferior incision in the thoracic cavity of the patient at a location inferior to the fracture in the rib; drilling through the first superior incision a first hole in the first bone segment of the rib; drilling through the second superior incision a second hole in the second bone segment of the rib; inserting a bone plate movably coupled to a single elongated flexible element through the third inferior incision to a location adjacent the fracture in the rib; inserting a first end of the single elongated flexible element through the first hole in the first bone segment and out through the first superior incision in a posterior-anterior direction relative to the rib; inserting a second end of the single elongated flexible element through the first hole in the second bone segment and out through the second superior incision in the posterior-anterior direction relative to the rib; pulling the first end and the second end of the single elongated flexible element in the posterior-anterior direction so as to 1) place the bone plate adjacent an anterior side of the rib across the fracture, 2) position first and second fasteners through the first and second holes, respectively; securing a first and second locking caps on the first and second fasteners, respectively; and pulling the single elongated flexible element in an anterior-posterior direction that is opposite the posterior-anterior direction to remove the elongated flexible element from the first fastener and the second fastener.

In Example 118, a bone fixation and repair system can be provided that includes a clamping device and a drill guide. The clamping device can be elongated along a longitudinal direction and can have: an articulating end including a movable holder configured to transition between an insertion configuration, where the movable holder aligned with the longitudinal direction, and articulated configuration, where the movable holder is offset with respect to the longitudinal direction, and an actuation end spaced from the articulating end along the longitudinal direction, the actuation end including a support member with a channel, and an actuator configured to transition the movable holder between the insertion configuration and the articulated configuration. The drill guide can be sized to fit in the channel and can have a distal end, a proximal end, and a lumen that extends from the distal end to the proximal end, wherein when 1) the drill guide is fully seated in the support member of the clamping device, and 2) the movable holder is in the articulated configuration, the distal end of the drill guide and the movable holder define a space sized to hold the bone therein.

In Example 119, the bone fixation and repair system of Example 118 can optionally be configured such that the clamping device includes a longitudinal member, and a slide member that slidable relative to the longitudinal member, wherein the movable holder is pivotably coupled to the longitudinal member and to the slidable member.

In Example 120, the bone fixation and repair system of Example 118 or Example 119 can optionally be configured such that the actuation end includes a trigger configured to permit the slide member to move along the longitudinal member, thereby cause the movable holder to pivot into the articulated configuration.

In Example 121, the bone fixation and repair system of any one or any combination of Examples 118-120 can optionally be configured such that the support member is threaded along the channel, and the proximal end of the drill guide is threaded to threadably engage the support member.

In Example 122, the bone fixation and repair system of any one or any combination of Examples 118-121 can optionally be configured to further include a drill configured to fit within the drill guide and drill a hole in the bone clamped by the movable holder of the clamping device.

In Example 123, the bone fixation and repair system of any one or any combination of Examples 118-122 can optionally be configured to further include: a bone plate having at least one opening, wherein the bone plate is bioabsorbable; at least one elongated flexible element; and a fastener assembly configured to fit within the at least one opening, the fastener assembly having a) a fastener and b) a locking cap configured to engage the fastener, the fastener including a channel that receives the at least one elongated flexible element.

In Example 124, a bone fixation and repair system can be provided that includes a bone plate, at least one elongated flexible element, and a fastener assembly. The bone plate can have at least one opening, wherein the bone plate can be bioabsorbable. The fastener assembly can be configured to fit within the at least one opening, the fastener assembly having a) a fastener and b) a locking cap configured to engage the shaft of the fastener, wherein at least one of 1) the fastener, 2) the at least one elongated flexible element, and 3) the locking cap are bioabsorbable.

In Example 125, the system or method of any one of or any combination of Examples 1-124 is optionally configured such that all elements or options recited are available to use or select from.

While the disclosure is described herein using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to a sequential series of reference signs and progression of the blocks in the Figures, the method can be implemented in a particular order, as desired.

What is claimed:

1. A bone fixation and repair system for a bone, comprising:
a bone plate elongated along a central longitudinal axis, the bone plate having a first end, a second end spaced from the first end along the central longitudinal axis, a first and second side member on opposite sides of the central longitudinal axis, and an elongated slot that extends through the bone plate along a depth that is perpendicular to the central longitudinal axis, the first side member and second side member each defining a bottom surface, a top surface spaced from the bottom surface along the depth, and an internal engagement surface that extends from the bottom surface toward the central longitudinal axis.

2. The bone fixation and repair system of claim 1, wherein the internal engagement surfaces are curved.

3. The bone fixation and repair system of claim 1, wherein the internal engagement surfaces are flat.

4. The bone fixation and repair system of claim 1, wherein the bone plate is curved along the central longitudinal axis.

5. The bone fixation and repair system of claim 1, wherein the internal engagement surfaces a) are non-orthogonal to the depth and b) are opposed to each other to partially define the elongated slot.

6. The bone fixation and repair system of claim 1, wherein the internal engagement surfaces extend along an entirety of the first side member and the second side member.

7. The bone fixation and repair system of claim 1, wherein the bone plate defines a) a length that extends from the first end to the second end, wherein the length is perpendicular to the depth, and b) a width that extends from the first side member to the second side member, wherein the width is perpendicular to the length and the depth, wherein the elongated slot extends along an entirety of the length.

8. The bone fixation and repair system of claim 1, wherein the top surface of the first side member and the second side member are curved in order to receive a curved surface of a bone segment.

9. The bone fixation and repair system of claim 1, wherein the first and second side members include first and second ridges along the top of the first and second side members, respectively.

10. The bone fixation and repair system of claim 1, wherein the elongated slot is a first elongated slot, and the bone plate further comprises a second elongated slot.

11. The bone fixation and repair system of claim 1, wherein the elongated slot is one of a plurality of elongated slots, wherein the plurality of elongated slots have a different size and orientation with respect to each other.

12. The bone fixation and repair system of claim 11, wherein bone plate further comprises at least two openings that extend through the bone plate along the depth.

13. The bone fixation and repair system of claim 1, wherein bone plate further comprises at least two openings that extend through the bone plate along the depth.

14. The bone fixation and repair system of claim 1, wherein the elongated slot is a first elongated slot, and the bone plate comprises:
a first segment including the first side member, the second side member, and the first elongated slot;
a second segment including a first side member, a second side member, a second elongated slot, where the first side member and the second side member of the second segment is substantially similar to the first side member and the second side member of the first segment;
a bridge segment that couples the first segment to the second segment, the bridge segment defining a bridge slot that is open the first and second elongated slots, such that a tethered fastener assembly is movable along any portion of the first elongated slot, the second elongated slot, and the bridge slot.

15. The bone fixation and repair system according to claim 1, further comprising:
a plurality of a tethered fastener assemblies configured to concurrently fit within and slide along any portion of the elongated slot, each tethered fastener assembly having 1) a fastener having a head, a shaft that extends from the head along a central fastener axis, and an elongated flexible element that is attached to and extends from the shaft, and 2) a locking cap configured to slide along the elongated flexible element into engagement with the shaft, wherein the head defines a distal end of the tethered fastener assembly and the elongated flexible element defines a proximal end of the assembly such that the tethered fastener assembly is an integrated unit.

16. The bone fixation and repair system of claim 15, wherein the head defines a smooth outer surface configured to movably engage the internal engagement surfaces of the bone plate, such that the fastener is rotatable about multiple axes when the smooth outer surface of the head is in contact the internal engagement surfaces of the bone plate.

17. The bone fixation and repair system of claim 16, wherein the smooth outer surface of the head is curved with respect to the central fastener axis.

18. The bone fixation and repair system of claim 16, wherein the smooth outer surface of the head is linear and angled with respect to the central fastener axis.

19. A bone fixation and repair system for a bone, comprising:
a bone plate configured to be placed against a bone, the bone plate including at least one opening; and
a plurality of a tethered fastener assemblies configured to secure the bone plate to the bone, each tethered fastener assembly having:
a) a fastener having a head, a shaft that extends from the head along a central fastener axis, and an elongated flexible element attached to and extending from the shaft, the shaft defining external ratchet teeth, wherein the head defines a distal end of the assembly and the elongated flexible element defines a proximal end of the assembly such that the tethered fastener assembly is an integrated unit, and
b) a locking cap configured to be attached to the fastener, the locking cap including a top, a bottom, an inner surface, and an inner opening defined by the inner surface, the inner surface defining internal ratchet teeth, wherein the internal ratchet teeth are configured to engage the external ratchet teeth of the shaft so as to lock the locking cap to the fastener.

20. A bone fixation and repair system for a bone, comprising:
a bone plate configured to be placed against a bone, the bone plate including at least one opening;
at least one tethered fastener assembly configured to extend through the at least one opening, the at least one tethered fastener assembly having:
a) a fastener having a head, a shaft that extends from the head along a central fastener axis, and an elongated flexible element attached to and extending from the shaft, the shaft having 1) a distal portion adjacent the head and having at least one engagement feature, and 2) a proximal portion adjacent to the distal portion such that the distal portion extends from the head to the proximal portion; and
b) a locking cap configured to engage the proximal portion of the shaft but not the distal portion of the shaft.
